(19) United States
(12) Patent Application Publication
(10) Pub. No.: US 2018/0232486 A1
(43) Pub. Date: Aug. 16, 2018

(54) QEEG/Genomic Analysis For Predicting Therapeutic Outcome

(71) Applicant: MYnd Analytics, Inc., Mission Viejo, CA (US)

(72) Inventors: George Carpenter, Laguna Nigel, CA (US); Brian MacDonald, Minneapolis, MN (US)

(21) Appl. No.: 15/893,080

(22) Filed: Feb. 9, 2018

Related U.S. Application Data
(60) Provisional application No. 62/457,560, filed on Feb. 10, 2017.

(51) Int. Cl. G06F 19/24; C12Q 1/6883; G01N 33/50; C12Q 1/26; A61B 5/0476; A61B 5/04; A61B 10/02; G16H 20/30; G06F 17/30; G06N 99/00

(52) U.S. Cl. CPC G06F 19/24; C12Q 1/6883; G01N 33/5067; C12Q 1/26; A61B 5/0476; G01N 2800/52; A61B 10/02; G16H 20/30; G06F 17/30283; G06N 99/005; C12Q 2600/106; A61B 5/04012

(57) ABSTRACT

Using a combinatorial algorithm comprised of quantitative EEG features and at least one pharmacogenomic variable, a significantly higher predictive accuracy and usability is achieved as compared to other current methods of clinical decision support for guided pharmacotherapy. The method produces a report with actionable findings for the treating physician, recommending for and/or against multiple drug classes and agents from among the available treatments for mental health disorders. While predictive accuracy for pharmacogenomic testing averages 73%, the presently disclosed combinatorial algorithms achieve a significantly higher rate of accuracy at 91%.

### FIELD OF THE INVENTION

The present invention is related to the processing an use of electroencephalographic data to predict susceptibility of individuals to psychiatric therapies. In particular, the process utilizes quantitative electroencephalographic analysis (QEEG) in combination with pharmacogenomic analysis. While the pharmacogenomic analysis can be performed using data from a gene set (e.g., more than one gene), improved accuracy of therapeutic efficacy predications is provided by using single gene data in combination with a QEEG analysis. For example, QEEG analysis therapeutic predictions of therapy response are materially improved by combination with pharmacogenetic analysis (PGx) and response trajectory (RT), used in sequence; QEEG→PGx→RT.

### BACKGROUND

The field of Psychiatry has long needed a physiology-based, repeatable, objective measure that correlates to medication response to inform clinicians in selection of psychotropic medications for their patient. Psychiatry is perhaps the only field of medicine where there is no recognized objective data to aid in diagnosis or medication selection. The standard for the field is the Diagnostic and Statistical Manual (need publisher) (DSM) which represents clusters of clinical symptoms. However these symptom clusters are relatively poor predictors of eventual medication response (need references and a good way to make this point). The DSM has been described as doing a good job of ensuring a common terminology among clinicians and researchers but a relatively poor job of informing treating physicians of likely responses to medications and other forms of treatment:
The strength of each of the editions of DSM has been “reliability”—each edition has ensured that clinicians use the same terms in the same ways. The weakness is its lack of validity. Unlike our definitions of ischemic heart disease, lymphoma, or AIDS, the DSM diagnoses are based on a consensus about clusters of clinical symptoms, not any objective laboratory measure.
   Thomas Insel, Director of the National Institutes of Mental Health (NIMH) (2103). As a result, a patient's chance of achieving response or remission of their symptoms when medications are selected based solely on DSM diagnosis is relatively poor.

What is needed in the art is a method to predict the efficacy of therapeutic success based upon a patients electroencephalgraphic and genomic characteristics because; i) no single gene is predictive; ii) the art has not provided any robust findings from only using QEEG features—due to ICA; iii) no a priori hypotheses has been selected from EEG features to create an improved drug efficacy prediction classifier.

### SUMMARY

The present invention is related to the processing an use of electroencephalographic data to predict susceptibility of individuals to psychiatric therapies. In particular, the process utilizes quantitative electroencephalographic analysis (QEEG) in combination with pharmacogenomic analysis. While the pharmacogenomic analysis can be performed using data from a gene set (e.g., more than one gene), improved accuracy of therapeutic efficacy predications is provided by using single gene data in combination with a QEEG analysis. For example, QEEG analysis therapeutic predictions of therapy response are materially improved by combination with pharmacogenetic analysis (PGx) and response trajectory (RT), used in sequence; QEEG→PGx→RT.

In one embodiment, the present invention contemplates a method, comprising: a) collecting an electroencephalogram and a plurality of cells from a patient exhibiting at least one symptom of a diagnosed psychiatric disorder; b) converting said electroencephalogram into at least one quantitative electroencephalographic (QEEG) feature variable; c) identifying at least one genotype in said tissue biopsy; d) comparing said at least one QEEG feature variable to a first database to create a first therapy list prioritized according to a first predicted efficacy score, said first therapy list comprising a first recommended therapy; e) comparing said at least one genotype (or a single genotype) to a second database to create a second therapy list prioritized according to a second predicted efficacy score, said second therapy list comprising a second recommended therapy; f) matching said first therapy list and said second therapy list to create a final therapy list prioritized according to a combined first and second efficacy score, said final therapy list comprising a final recommended therapy; and g) administering said final recommended therapy to said patient under conditions such that said at least one symptom is reduced, wherein said selected therapy comprises a combined first and second efficacy score that is within a preferred range. In one embodiment, said final recommended therapy is different from said first recommended therapy and said second recommended therapy. In one embodiment, said first recommended therapy and said second recommended therapy are the same. In one embodiment, said first recommended therapy and said second recommended therapy are different. In one embodiment, said plurality of cells is derived from a patient biopsy.

In one embodiment, the present invention contemplates a method, comprising: a) collecting an electroencephalogram and a plurality of cells from a patient exhibiting at least one symptom of a diagnosed psychiatric disorder; b) converting said electroencephalogram into at least one quantitative electroencephalographic (QEEG) feature variable; c) comparing said at least one QEEG feature variable to a first database to identify a prioritized list of recommended drugs; d) processing said prioritized list of recommended drugs with an in vitro enzyme metabolism assay using said plurality of cells derived from said tissue biopsy to identify a list of recommended drugs prioritized by metabolic rate; e) selecting a preferred recommended drug by identification of a non-metabolic drug biomarker in said tissue biopsy that matches at least one drug on said metabolic rate prioritized list of recommended drugs; f) administering said preferred recommended drug to said patient under conditions such that said at least one symptom is reduced. In one embodiment, the non-metabolic drug biomarker is a blood based biomarker. In one embodiment, the non-metabolic drug biomarker is a cell based biomarker. In one embodiment, said plurality of cells is derived from a patient biopsy.

In one embodiment, the present invention contemplates a method, comprising: a) collecting an electroencephalogram and a plurality of cells from a patient exhibiting at least one symptom of a diagnosed psychiatric disorder; b) converting said electroencephalogram into at least one quantitative electroencephalographic (QEEG) feature variable, said QEEG feature variable having a predetermined drug efficacy predictive value; c) identifying at least one genotype (or a single genotype) in said plurality of cells, said at least one genotype having a predetermined drug efficacy predictive value; d) combining said QEEG feature variable predetermined drug efficacy predictive value and said at least one genotype predetermined drug efficacy predictive value to create a list of recommended drugs prioritized by an efficacy score; and e) administering at least one of said recommended drugs to said patient under conditions such that said at least one symptom is reduced, wherein said efficacy score of said selected drug is within a preferred range. In one embodiment, said plurality of cells is derived from a patient biopsy.

In one embodiment, the present invention contemplates a method, comprising: a) collecting an electroencephalogram and a plurality of cells from a patient exhibiting at least one symptom of a diagnosed psychiatric disorder; b) converting said electroencephalogram into at least one quantitative electroencephalographic (QEEG) feature variable; c) comparing said at least one QEEG feature variable to a first database to identify a prioritized list of recommended drugs; d) processing said prioritized list of recommended drugs with at least one metabolic genotype (or a single metabolic genotype) using said plurality of cells to identify a list of said recommended drugs prioritized by metabolic rate; e) selecting a preferred recommended drug by identification of a non-metabolic drug biomarker in said plurality of cells that matches at least one drug on said metabolic rate genotype prioritized list of recommended drugs; f) administering said preferred recommended drug to said patient under conditions such that said at least one symptom is reduced. In one embodiment, said plurality of cells is derived from a patient biopsy.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “substitute for” as used herein, refers to the switching the administration of a first compound or drug to a subject for a second compound or drug to the subject.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “genotype” as used herein, refers to any nomenclature that identifies the particular genetic composition of a defined nucleic acid sequence within a patient. For example, a genotype may refer to any one of several alleles of a single gene. Alternatively, a genotype may also refer to a specific sequence of genes arranged, in order, on a patient's chromosome. Identification of such genotypes may be determined by methods known in art including, but not limited to, nucleic acid sequences and/or single nucleotide polymorphisms (SNPs).

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of, the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, symptoms of a patient may include, but are not limited to behavioral symptoms such as those persistent or repetitive behaviors that are unusual, disruptive, inappropriate, or cause problems. More specifically, actions including, but not limited to, aggression, criminal behavior, defiance, drug use, hostility, inappropriate sexual behavior, inattention, secrecy, and/or self-harm are considered behavioral symptoms. In conventional clinical psychiatric practice, diagnoses are highly dependent upon the presence or absence of behavioral symptoms as indexed in the DSM-IV. For example, psychriatric symptoms may include, but are not limited to, inappropriate behavior, inappropriate emotions, learning disorders, difficulty in interpersonal relationships, general unhappiness, unexplained fear, unexplained anxiety, insomnia, irrational thoughts, obsessions, compulsions, easily annoyed, easily nervous, unexplained anger, unnecessarily blaming others and/or substance abuse. Alternatively, subjective evidence of an untreated behavioral disorder is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “sample” or “biopsy” as used herein is used in its broadest sense and includes environmental and biological samples. Such samples and/or biopsies may contain a plurality of cells, from a subject or patient's tissues. Such tissues may include, but are not limited to, liver tissues, buccal tissues, bone marrow tissues, skin tissues etc. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents a graphic of one embodiment of a QEEG/Genomic therapeutic prediction algorithm. The results plot specific therapies in accordance to their categories from a genomic analysis: i) Consider Alternatives (red); ii) Use With Caution (yellow); and iii) Standard Precautions (Green); and a predictive factor of likely success using QEEG analysis; i) Not As Likely (red); ii) Moderately Likely (White); and iii) Likely (Blue). Therapies in the Green genomic category and Blue QEEG category are the best candidates, while therapies in the Red genomic category and Red QEEG category are the worst candidates.

FIG. 2 presents one embodiment of an improved drug efficacy classification algorithm that operates based upon machine learning.

FIG. 3 presents exemplary data of in silico therapeutic efficacy predictions with a combination of QEEG feature variable data and quantitated genotype data from a single gene.

FIG. 4 A-C present exemplary data showing stable correlation patterns between QEEG feature variables and a positive patient outcome or a negative patient outcome.

FIG. 4A: Each unshaded “box” (e.g., analysis bin) represents one treatment interval that meets the inclusion criteria for analysis.

FIG. 4B: Overlays FIG. 4A with patients having a positive outcome to treatment are shown as light shaded boxes. The solid light shaded curved line represents the overall distribution of positive responders over the treatment period,

FIG. 4C. Overlays FIGS. 4A and 4B with patients having a negative outcome (e.g., a non-response) to treatment are shown as dark shaded boxes. The solid dark shaded curved line represents the overall distribution of non-responders over the treatment period.

FIG. 5 shows a representative PEER report showing the distribution of responders (blue) and non-responders (red) to fluoxetine having a similar QEEG feature variable pattern as the patient (X).

FIG. 6 A-B shows the basis for the commerically available GeneSight® genomic analysis for predicted therapeutic response.

FIG. 6A: Identifies the specific six (6) genes that comprise the “composite phenotype” to determine a risk categorization of each drug considered for administration.

FIG. 6B: Shows a representative GeneSight report that categorizes antidepressants and antipsychotics in specific risk categories without any prioritization regarding their respective predicted therapeutic efficacy,

FIG. 7 A-D presents exemplary summary data from several randomized, double-blinded controlled trials of PEER and predecessor rEEG studies, as discussed herein, where PEER guidance was compared to Treatment As Usual (TAU) in the treatment of patients with Treatment-Resistant Depression (TRD).

FIG. 7A: Veterans Administration—Sepulveda (J Am Physicians & Surgeons, 2007).

FIG. 7B: Depression Efficacy Pilot Study12 (NCDEU, 2009).

FIG. 7C: Depression Efficacy Study—Harvard/Stanford multi-site (J Psych Res, 2011).

FIG. 7D: Walter Reed PEER interactive Trial—(Neuropsychiatric Disease and Treatment, 2016).

FIG. 8 presents a representative flow chart of a QEEG/Genomic analysis evaluation design.

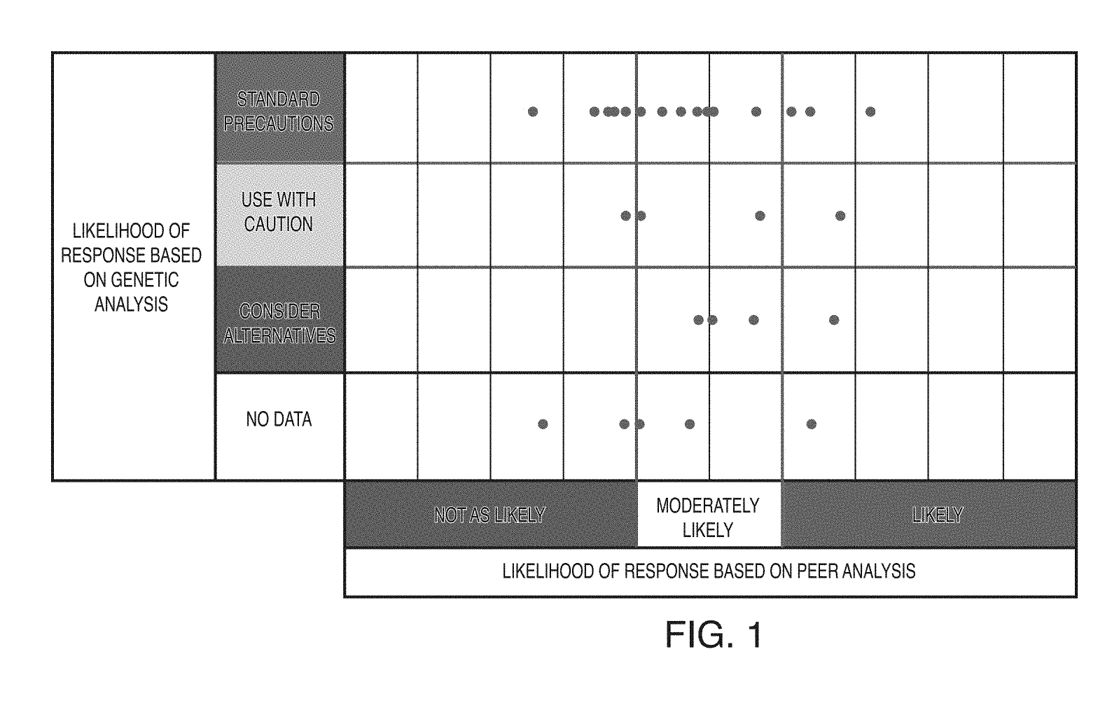

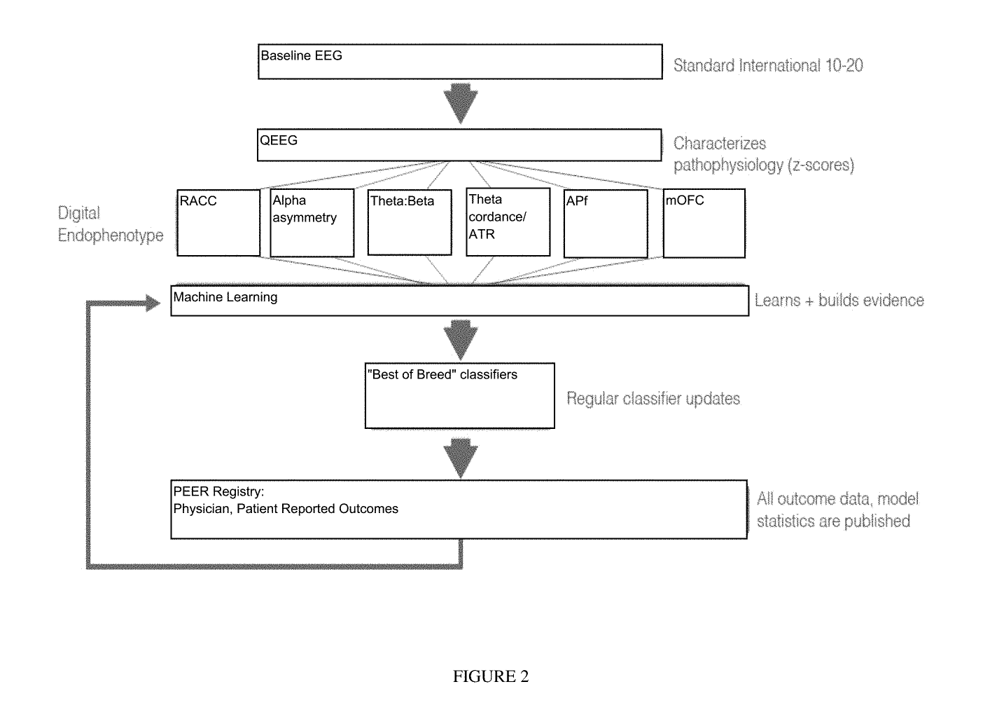

### DETAILED DESCRIPTION OF THE INVENTION

The present invention is related to the processing an use of electroencephalographic data to predict susceptibility of individuals to psychiatric therapies. In particular, the process utilizes quantitative electroencephalographic analysis (QEEG) in combination with pharmacogenomic analysis. While the pharmacogenomic analysis can be performed using data from a gene set (e.g., more than one gene), improved accuracy of therapeutic efficacy predications is provided by using single gene data in combination with a QEEG analysis. For example, QEEG analysis therapeutic predictions of therapy response are materially improved by combination with pharmacogenetic analysis (PGx) and response trajectory (RT), used in sequence; QEEG→PGx→RT.

In some embodiments, the present invention combines a large clinical outcome registry with personal physiological data (electrophysiology and pharmacogenomic data) and machine learning to improve the accuracy of prescribing in mental health. Although it is not necessary to understand the mechanism of an invention, it is believed that large clinical outcome registries, as contemplated herein, are highly structured and may be managed by conventional database software including, but not limited to, MSSQL, Oracle, MySQL etc. it is further believed that these database software programs are compatible with the presently disclosed methods of collecting and analyzing data to identify and improve drug treatment efficacy. In one embodiment, the present method selects psychotropic medications (e.g., for example, antidepressants) based on a subjective, non-biological, scientifically invalid diagnostic taxonomy and a trial and error procedure for medication selection.

Certain embodiments of the present invention contemplate methods comprising genotyping at least one gene to predict drug efficacy in a patient. In one embodiment, the method may comprise genotyping a single gene without having to genotype a second gene. In other embodiments, the method may comprise genotyping a plurality of genes, wherein the accuracy of predicting the drug efficacy is improved over that of any one of the single genes. The steps within the method embodiments disclosed herein may be performed in any order. However, it is preferred that the step of determining either the metabolic drug genotype or a drug metabolic rate of a patient be performed first. Although it is not necessary to understand that mechanism of an invention, it is believed that by determining the metabolic drug status of a patient first, will quickly eliminate useless drugs quickly, such that the further steps of using QEEG predictors and non-metabolic predictors may be assessed in a condition where the signal-to-noise ratio is greatly enhanced.

### I. Current Electroencephalographic Methods of Predicting Therapeutic Efficacy

Objective data and methods for medication selection have long been sought, but methods for predicting treatment response based on individual physiology have had limited demonstrated efficacy and adoption. Quantitative electroencephalography (QEEG) has shown some limited success in predicting medication selection, but one of the largest clinical trials using quantitative EEG demonstrated a very limited correlation between EEG and quantitative EEG features. Similarly, predictive pharmacogenomic findings have been focused upon drug availability by monitoring pharmacokinetics (e.g., drug metabolism), which affects a limited segment of the population. The findings of these methods are complementary, in that they address different body systems and treatment pathways, but to date neither has been combined in a clinical decision algorithm.

One early study comprised a prospective, blinded, controlled design that compared outcomes in chronic, refractory major depressive disorder (MDD) with and without physicians' prescribing medications guided by electroencephalography-based medication outcome prediction. There were statistically significant differences between the two groups in pretreatment vs. treatment Hamilton Depression Scale (HAM-D) and Beck Depression Inventory scores (P<0.009) and Clinical Global Impression (CGI) scores (P=0.02). Only one of six patients demonstrated clinical improvement with medication choice unguided by EEG data, compared to six of seven treated with EEG guidance. Pretreatment EEG data predicted medication response in this pilot study. Suffin et al., “A QEEG Database Method for Predicting Pharmacotherapeutic Outcome in Refractory Major Depressive Disorders” J. Am. Phys. Surg. 12(4)104-108 (2007).

Recent results from the NIMH study entitled “Sequenced Treatment Alternatives to Relieve Depression” (STAR*D) suggest that a patient diagnosed with depression has a 37% chance of experiencing remission and a 47% chance of experiencing a response on their first treatment episode. These remission and response rates dropped significantly at each step of treatment to 13.0% for remission and response after 3 unsuccessful medication trials. Relapse and dropout rates also increase at each treatment failure (Rush, et. al., 2006, Warden, et. al. 2007).

There have also been attempts to develop objective, physiology-based markers in mental illness based on a variety of modalities. For example, genetic information as an indicator of diagnosis and medication response is being promoted by several commercial enterprises. Genomind, 100 Highpoint Drive, Suite 102, Chalfont, Pa. 18914, genomind.com. Other modalities being investigated include fMRI, PET, SPECTS, and biomarkers present in the blood and/or other tissues. Oliveira et. al., “What does brain response to neutral faces tell us about major depression? evidence from machine learning and fMRI” PLoS One 8(4):e60121 (2013); and Fatemi et al., “Altered levels of Reelin and its isoforms in schizophrenia and mood disorders” Neuroreport. 12(15):3209-3215 (2001).

One of the modalities that continues to receive attention from researchers and clinicians is QEEG. In addition to being indicative of the functional connectivity of the brain, the electroencephalogram (EEG), from which QEEG is derived, is inexpensive, non-invasive and can be administered in an office, home or hospital in-patient setting. Tan et al., “The Difference of Brain Functional Connectivity between Eyes-Closed and Eyes-Open Using Graph Theoretical Analysis” Comput Math Methods Med 2013:976365; and De La Fuente et al., “A review of attention-deficit/hyperactivity disorder from the perspective of brain networks” Front Hum Neurosci. 7:192 (2013). QEEG, unlike visual interpretation of the EEG, shows a high level of repeatability and consistency over time. Malone, et. al. (2009); Gerber, et. al. (2008); and Napflin, et. al. (2007). Another benefit of QEEG is the existence of large repositories of the EEG of asymptomatic individuals and a large body of research on the effects of medications on the EEG and resulting QEEG. Although it is not necessary to understand the mechanism of an invention it is believed that QEEG results from the frequency decomposition of the digital EEG to yield more information from the EEG than is obtainable from visual inspection. For example, QEEG is intended to augment visual inspection of the EEG but not to replace it.

Several QEEG analysis software tools have been cleared by the FDA for the post-hoc statistical evaluation of the human EEG. Warden et al., “The STAR*D Project results: a comprehensive review of findings” Curr Psychiatry Rep. 9(6):449-459 (2007); and Anonymous, “Neuroguide Analysis System” Applied Neuroscience, Inc., 228 176th Terrace Drive, St. Petersburg, Fla. 33708, accessdata.fda.gov/cdrh_docs/pdf4/k041263.pdf. These software tools also contain, as part of their analysis, comparison to age-matched norms. The existence of these norms provides a control group from which patterns of abnormalities in the EEG can be assessed and categorized. Thatcher et al., “History of the Scientific Standards of QEEG Normative Databases” In: Budzynski, et. al. Introduction to Quantitative EEG and Neurofeedback (Second Edition), Elsevier, Inc., 2009, Chapter 2.

Most neurometric features are highly non-Gaussian in their characteristics. For this reason, commercially available neurometric software transforms the raw data using a log transform to make the distributions more Gaussian in nature. Many quantitative EEG features also vary consistently with age. To account for the difference between the age of the patient and the age of the subjects in the normative database, these quantitative EEG features are age-regressed using a linear regression equation to yield a “standard-age” quantitative EEG feature. These measures include, but are not limited to:
Absolute power—the power, expressed in uV2, of the EEG waveform within each of the frequency bands
Relative power—the percentage of absolute power in each of the bands
Mean frequency—the average of the component frequencies within each of the frequency bands. Examining the average frequency can provide information on whether the component frequencies are clustered toward one side of the frequency band.
Coherence—the degree of synchronization of electrical activity between two channels and is often interpreted as a measure of the functional association between two areas of the brain. Srinivasan et al., “EEG and MEG coherence: measures of functional connectivity at distinct spatial scales of neocortical dynamics” J Neurosci Methods. 166(1): 41-52 (2007).
Symmetry—a measure of the difference in the amplitude between two electrodes. Certain patterns of symmetry have been hypothesized as markers for depression and schizophrenia. Stewart et al., “Resting Frontal EEG Asymmetry as an Endophenotype for Depression Risk: Sex-specific Patterns of Frontal Brain Asymmetry” J Abnorm Psychol. 119(3): 502-512 (2010); and Merrin et al., “EEG asymmetry in schizophrenic patients before and during neuroleptic treatment” Biol Psychiatry 21(5-6):455-464 (1986).

Remission rates for Major Depressive Disorder (MDD) are low and unpredictable for any given antidepressant and no biological or clinical marker has demonstrated sufficient ability to match individuals to efficacious treatment. Biosignatures developed from the systematic exploration of multiple biological markers, which optimize treatment selection for individuals (moderators) and provide early indication of ultimate treatment response (mediators) are needed.

One study provided a rationale and design of a multi-site, placebo-controlled randomized clinical trial of sertraline examining moderators and mediators of treatment response. The target sample was 300 participants with early onset (≤30 years) recurrent MDD. Non-responders to an 8-week trial were switched double blind to either bupropion (for sertraline non-responders) or sertraline (for placebo non-responders) for an additional 8 weeks. Clinical moderators include anxious depression, early trauma, gender, melancholic and atypical depression, anger attacks, Axis II disorder, hypersomnia/fatigue, and chronicity of depression. Biological moderator and mediators included cerebral cortical thickness, task-based fMRI (reward and emotion conflict), resting connectivity, diffusion tensor imaging (DTI), arterial spin labeling (ASL), electroencephalograpy (EEG), cortical evoked potentials, and behavioral/cognitive tasks evaluated at baseline and week 1, except DTI, assessed only at baseline. The study was designed to standardize assessment of biomarkers across multiple sites as well as institute replicable quality control methods, and to use advanced data analytic methods to integrate these markers. Trivedi et al., “Establishing moderators and biosignatures of antidepressant response in clinical care (EMBARC): Rationale and design” J Psychiatr Res. 78:11-23 (2016). In particular, this study tested whether pre-treatment resting EEG alpha or theta measures and LDAEP moderate response to an SSRI. While the data collection for this study has been completed, the analysis and reporting of the results has yet to be completed.

Given the high prevalence of treatment-resistant depression and the long delays in finding effective treatments via trial and error, valid biomarkers of treatment outcome with the ability to guide treatment selection represent one of the most important unmet needs in mood disorders. A large body of research has investigated, for this purpose, biomarkers derived from electroencephalography (EEG), using resting state EEG or evoked potentials. Most studies have focused on specific EEG features (or combinations thereof), whereas more recently machine-learning approaches have been used to define the EEG features with the best predictive abilities without a priori hypotheses. Several measures derived from the analysis of spontaneous EEG, evoked potentials, and EEG source localization have been associated with antidepressant response and have the potential to offer relatively simple and inexpensive predictors of such response. Other studies have suggested that baseline QEEG parameters may also serve to predict the total burden of treatment-emergent side effects or more specifically to predict treatment-emergent suicidal ideation, which would enhance the clinical value of EEG biomarkers. Hunter et al. (2005): Neurophysiologic correlates of side effects in normal subjects randomized to venlafaxine or placebo. Neuropsychopharmacology 30:792-799: Iosifescu et al., (2008): Pretreatment frontal EEG and changes in suicidal ideation during SSRI treatment in major depressive disorder. Acta Psychiatr Scand 117:271-276; Hunter et al., (2010): Brain functional changes (QEEG cordance) and worsening suicidal ideation and mood symptoms during antidepressant treatment. Acta Psychiatr Scand 122:461-469; and Iosifescu DV (2011): Electroencephalography-derived biomarkers of antidepressant response. Harv Rev Psychiatry 19: 144-154.

Notably, the iSPOT-D study did not yield general statistically significant results, besides the CNS-arousal and genderspecific alpha asymmetry findings. This could indicate that some of the EEG biomarkers investigated represent models that were overfit to their datasets. Based on the large volume of work on this topic, it is important to understand how the different measures discussed here relate to each other. However, at this point there does not seem to be consistent data on the relationship between different measures. Additionally, most studies present unique combinations of EEG features, which prevent a coherent explanatory model or meta-analytic approaches. Even the biomarkers most advanced in development, theta cordance and ATR, leave unanswered many questions related to their usefulness: the values used to define nonresponse differ across studies; results may be valid only for the antidepressants tested (mostly SSRIs, serotonin and norepinephrine reuptake inhibitors, and bupropion); and it is unclear how predictors could be combined with clinical and other measures for improved predictive accuracy. The most promising avenue for future development is represented by studies using machine learning, which will enable processing of large databases (including clinical datasets from electronic medical records) to validate predictors and test their clinical usefulness. Wade et al., “Using EEG for Treatment Guidance in Major Depressive Disorder” Biological Psychiatry: Cognitive Neuroscience and Neuroimaging, 1(5): 411-422 (2016).

### II. Therapy/EEG Interactions

That medications can alter the EEG has been observed by visual and quantitative electroencephalographers alike. Medication-induced changes in the QEEG have been reported for a broad range of antidepressants, benzodiazepines, stimulants, antipsychotics, lithium salts, and anticonvulsants. Thau et al. “Effect of lithium on the EEG of healthy males and females. A probability mapping study” Neuropsychobiology 20(3):158-163 (1989); Salinsky et al., “Assessment of CNS effects of antiepileptic drugs by using quantitative EEG measures” Epilepsia 44(8); 1042-1050 (2003); Hardmeier et al., “Intranasal midazolam: pharmacokinetics and pharmacodynamics assessed by quantitative EEG in healthy volunteers” Clin Pharmacol Ther. 91(5):856-862 (2012); Kerdar et al. “Quantitative effect of treatment with methylphenidate on EEG—a pilot study” Z Kinder Jugendpsychiatr Psychother. 35(4):247-255 (2007); and Clemens et al., “Quantitative EEG effects of carbamazepine, oxcarbazepine, valproate, lamotrigine, and possible clinical relevance of the findings” Epilepsy Research 70(2-3):190-199 (2006).

These drug changes are specific in regard to effects on distinct components of the QEEG and consistent in their effect in subjects with psychiatric syndromes as well as asymptomatic volunteers. If medications alter the human EEG in consistent and known ways, then it may be possible to use this information to inform clinical treatment decisions by using these medication effects to counteract observable QEEG patterns in patients. For example, a study of 70 patients with major affective disorders demonstrated that an array of QEEG variables were more predictive of response to paroxetine at 6 weeks of followup than were pre-treatment HAM-D scores. Knott, et. al. (2000). For example, a study of 82 subjects meeting DSM criteria for Major Depressive Disorder found that baseline relative theta power at baseline (pre-treatment) predicted treatment response to SSRI's or venlafaxine with 63% accuracy and that an Antidepressant Treatment Response (ATR) index comprised of a variety of QEEG variables was predictive of SSRI or venlafaxine with 70% accuracy. Iosefescu et al. (2009).

These studies also found that there were significant QEEG heterogeneities within neuropsychiatric diagnoses. The existence of these subgroups within DSM diagnosis categories may help to explain the differential efficacy of drugs prescribed for these diagnoses and suggests that QEEG patterns may be able to play a role in assisting the clinician in choosing the most effective pharmacotherapy for a specific patient.

Brain imaging has been utilized in an effort to determine whether or not a particular therapy has an efficacious effect on a psychiatric disorder. Approximately 50% of patients with major depressive disorder (MDD) do not respond optimally to antidepressant treatments. Given this is a large proportion of the patient population, pretreatment tests that predict which patients will respond to which types of treatment could save time, money and patient burden. Brain imaging offers a means to identify treatment predictors that are grounded in the neurobiology of the treatment and the pathophysiology of MDD. The International Study To Predict Optimized Treatment in Depression (iSPOT-D) was a multi-center, parallel model, randomized clinical trial with an embedded imaging sub-study to identify such predictors. A focus was placed on brain circuits implicated in major depressive disorder and its treatment. In the full trial, depressed participants were randomized to receive escitalopram, sertraline or venlafaxine-XR (open-label). They were assessed using standardized multiple clinical, cognitive-emotional behavioral, electroencephalographic and genetic measures at baseline and at eight weeks post-treatment. Overall, 2,016 depressed participants (18 to 65 years old) entered the study, of whom a target of 10% will be recruited into the brain imaging sub-study (approximately 67 participants in each treatment arm) and 67 controls. Structural studies included high-resolution three-dimensional T1-weighted, diffusion tensor and T2/Proton Density scans. Functional studies included standardized functional magnetic resonance imaging (MRI) with three cognitive tasks (auditory oddball, a continuous performance task, and Go-NoGo) and two emotion tasks (unmasked conscious and masked non-conscious emotion processing tasks). After eight weeks of treatment, the functional MRI is repeated with the above tasks. Predictors were identified using half the subjects (n=102), while the second half were initially tested, with an overall analyses extending to all tested subjects. Grieve et al., “Brain imaging predictors and the international study to predict optimized treatment for depression: study protocol for a randomized controlled trial” Trials 14:224 (2013).

Preliminary data from the iSPOT study determined whether EEG occipital alpha and frontal alpha asymmetry (FAA) distinguishes outpatients with major depression (MDD) from controls, predicts antidepressant treatment outcome, and to explore the role of gender. No differences in EEG alpha for occipital and frontal cortex, or for FAA, were found in MDD participants compared to controls. Alpha in the occipital and frontal cortex was not associated with treatment outcome. However, a gender and drug-class interaction effect was found for FAA. Relatively greater right frontal alpha (less cortical activity) in women only was associated with a favorable response to the Selective Serotonin Reuptake Inhibitors escitalopram and sertraline. No such effect was found for venlafaxine-extended release. Arns et al., “EEG alpha asymmetry as a gender-specific predictor of outcome to acute treatment with different antidepressant medications in the randomized iSPOT-D study” Clin Neurophysiol. 127(1):509-519 (2016).

QEEG analysis has been refined and documented to function in conjunction with an outcomes database holding patient data including, but not limited to, QEEG multivariate patterns and response outcomes to specific therapies. This analysis has been give the trademark recognition of rEEG® to currently named company of MYnD Analytics, Inc. CNS Response, Inc. CNS response [online]. Available from: cnsresponse.com/doc/CNSR_rEEG_Intro_Guide_to_EEG_Recording_v2.0_Mar2009.pdf.

Referenced-EEG (rEEG®) provides evidenced-based medication guidance formulated on a set of empirically-derived Biomarkers used to guide psychopharmacologic treatment, primarily for treatment resistant cases. rEEG® employs a large database of unmedicated, pre-treatment quantitative EEGs (QEEGs) in patients with psychiatric symptomatology who were subsequently treated with a broad range of medications, while recording their clinical responses. This permits a correlation between EEG abnormality, medication and response. The average time to clinical response in this database was 405 days allowing the correlations to avoid efficacy anomalies such as placebo response. In one pilot study, rEEG-guidance was compared to guidance based on the Texas Medication Algorithm Project (TMAP) in the treatment of patients with Treatment-Resistant Depression (TRD) conducted at eight centers in the US, including several major academic institutions. The study was designed to compare 10-week treatment outcomes in patients who were medicated based on the TMAP depression algorithm versus patients who were medicated based on rEEG-guided options. This was a multicenter, randomized, blinded, controlled, parallel group study with 18 completers. Subjects had failed at least three prior antidepressant regimens of adequate dose for a minimum duration of 4 weeks. All subjects underwent a washout of all current medications for a minimum of 5 half-lives and then had a QEEG recorded and analyzed utilizing rEEG technology to determine a treatment recommendation. Subsequently qualified subjects were randomized to the experimental (rEEGguided) group, or the control group utilizing the TMAP. The results of this study have been reported previously. This post hoc analysis evaluates the subsets of subjects who would have had ‘equivalent’ recommendations for either the rEEG or the TMAP treatment groups.

When Pure rEEG-guided was compared to Pure TMAP-guided treatment (Sub-groups 1 vs 3) the results show those subjects receiving the rEEG-guided recommended treatments were different, not equivalent to TMAP-guided treatment. For example, responses in Q-LES-Q score, defined as an improvement by at least 25 points (there are no standardized definitions for response or remission), and was attained by 29% in the rEEG-treated group whereas none was observed in the TMAP-treated group. Similarly for QIDS, 57% and 43% of participants in the rEEG group reached response (at least 50% reduction) or remission (raw score <6), respectively, across the 10-week trial compared to none in the TMAP group. These data show that significantly more subjects who were treated with the rEEG-guided medication reached their respective response/remission targets, while none did in the pure TMAP group. DeBattista et al., “Review of Current Results in the Use of Referenced-EEG in the Guidance of Psychotropic Medication Selection for Treatment-Resistant Depressed Patients” NCDEU Poster (June 2009)

A recently reported study evaluated the efficacy of rEEG®-guided pharmacotherapy for the treatment of depression in those circumstances where rEEG and STAR*D provided different recommendations. A randomized, single-blind, parallel group, 12 center, US study compared rEEGguided pharmacotherapy vs. the most effective treatment regimens reported in the NIH sponsored STAR*D study. Relatively treatment-resistant subjects ≥18 years who failed one or more antidepressants were required to have a QIDS-16-SR score ≥13 and a MADRS score ≥26 at baseline. All subjects underwent a washout of all current medications (with some protocol-specified exceptions) for at least five half-lives before receiving a QEEG and rEEG report. Subjects randomized to rEEG were assigned a regimen based on the rEEG report. Control subjects who had failed only SSRI's in their current episode were randomized to receive venlafaxine XR. Control subjects who had failed antidepressants from ≥2 classes of antidepressants were randomized to receive a regimen from Steps 2-4 of the STAR*D study. Treatment lasted 12 weeks. The primary outcome measures were change from baseline for self-rated QIDS-SR16 and Q-LES-Q-SF. A total of 114 subjects were randomized and 89 subjects were evaluable. rEEG-guided pharmacotherapy exhibited significantly greater improvement for both primary endpoints, QIDS-SR16 (−6.8 vs. −4.5, p<0.0002) and Q-LES-Q-SF (18.0 vs. 8.9, p<0.0002) compared to control, respectively, as well as statistical superiority in 9 out of 12 secondary endpoints. These results suggest a role for rEEG-guided psychopharmacology in the treatment of depression and that rEEG-guided pharmacotherapy represents a predictive and objective office procedure that builds upon clinical judgment to guide antidepressant medication choice. DeBattista et al., “The use of referenced-EEG (rEEG) in assisting medication selection for the treatment of depression” Journal of Psychiatric Research 45(1):64-75 (2010).

### III. The Psychiatric Electroencephalographic Evaluation Registry (PEER)

The Psychiatric Electroencephalographic Evaluation Registry (PEER) is a QEEG derived tool that uses QEEG derived patterns of abnormalities and historical databases of patient outcomes to assist physicians in medication selection.

The PEER process can begin by collecting awake, digital EEG (i.e., for example, eyes-closed or eyes-open) that conforms to the international 10/20 standard. A variety of EEG collection hardware may be supported. Although it is not necessary to understand the mechanism of an invention, it is believed that such data collection protocols are compatible with conventional QEEG tools (e.g., Neuroguide) that generally compile eyes-open normal EEG data. Eligible patients are usually between the ages of 6 and 85 and are preferably medication-free or free of all medications that can affect the EEG, including but not limited to, naturopathic and herbal products and anything that crosses the blood/brain barrier, for at least five (5) half-lives. These criteria help to ensure compatibility with normative (e.g, for example, control) databases within most commercially available QEEG software programs.

Approximately ten (10) to twenty (20) minutes of raw EEG is sufficient in most cases. The EEG may then be manually edited to select at least two minutes of artifact-free EEG. Artifacts can include, but are not limited to, eye blinks and movement, muscle movement, drowsiness and/or others. It should be of interest that automatic artifact rejection techniques based on individual components analysis (ICA) that are currently available are not used in the PEER process. In parallel with this editing step, the raw EEG may be visually reviewed by an electroencephalographer for overall quality. For example, this quality review can ensure that no gross pathology is present such as seizure activity or encephalopathy.

One QEEG software that may be used in the present method is the Neuroguide® software which is approved by the FDA for “post-hoc statistical evaluation of the human EEG” (Applied Neuroscience, Inc., Largo, Fla., appliedneuroscience.com). The Neuroguide® Software provides support for most commercially available EEG machines and supports most EEG digital file formats. The Neuroguide® software also provides amplifier correction to account for the frequency response characteristics of the EEG amplifiers supported, age correction using a linear regression equation to yield a “standard-age” quantitative EEG feature and transformation of QEEG features to make them more gaussian in nature where necessary. After a suitable amount of artifact-free data is selected, the Neuroguide® software transforms the EEG waveforms by means of a Fast Fourier Transform (FFT) into its component frequencies. These component frequencies are then aggregated into frequency bands of; Delta (1.5 hz-3.5 hz), Theta (3.5 hz-7.5 hz), Alpha (7.5 hz-12.5 hz) and Beta (12.5 hz-25.5 hz). The Neuroguide® software then computes a series of measures from these frequency bands. These raw QEEG measures derived from the FFT decomposition of the EEG signal may then be compared to a normative database (e.g., for example, a database comprising EEG data from 625 asymptomatic subjects ranging in age from 2 months to 85 years). Thatcher et al. The differences between the actual values of the patient-derived neurometric variables with normative neurometic variables are expressed as Z scores.

A. Development of QEEG Feature Variables

Neurometric analysis using the Neuroguide® software upon which PEER is based outputs approximately 7,200 Z-scored individual variables that describe the EEG waveforms. To make this data utilizable, the PEER database transforms this Z-score data into a smaller set of QEEG feature variables (e.g., for example, a multivariable). In one embodiment, a QEEG feature variable preserves and reduces a set of QEEG univariate data while retaining some degree of physical interpretation. In one embodiment, the PEER database comprises at least two hundred and twenty-three (223) QEEG feature variables.

B. Correlation of Feature Variables with Known Patient Outcomes

In one embodiment, the presently contemplated method comprises correlating known patient outcomes with at least one QEEG feature variable. In some embodiments, the known patient outcome is correlated with a QEEG feature variable pattern, wherein the QEEG feature variable pattern comprises a plurality of different QEEG feature variables.

For example, patient EEG data may be recorded according to EEG protocols that ensure its comparability to the normative database within the QEEG software. Patients are then treated according to a DSM guided methodology and their outcomes to treatment are recorded. At periodic intervals during the treatment process patient outcome is quantitated using a Clinical Global Impression-Improvement (CGI-I) score along with the “treatment interval” of the medication (e.g., by determining the start and stop dates of medication administration). The original developers of the methodology that was improved to become a PEER database began collecting medication-free EEGs and recording patient outcomes to DSM guided treatment in the early 1990's. Suffin et al., “Electroencephalography Based Systems and Methods for Selecting Therapies and Predicting Outcomes” PCT/US2002/021976.

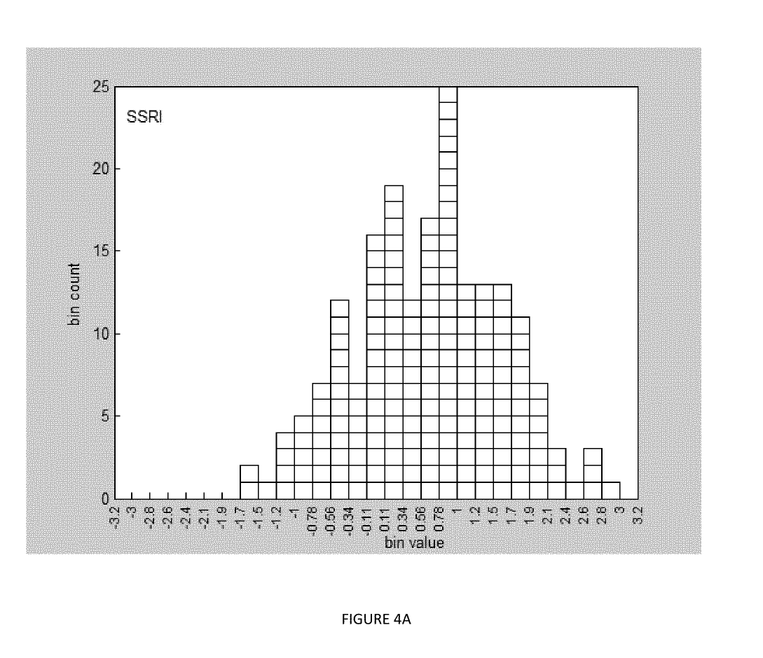

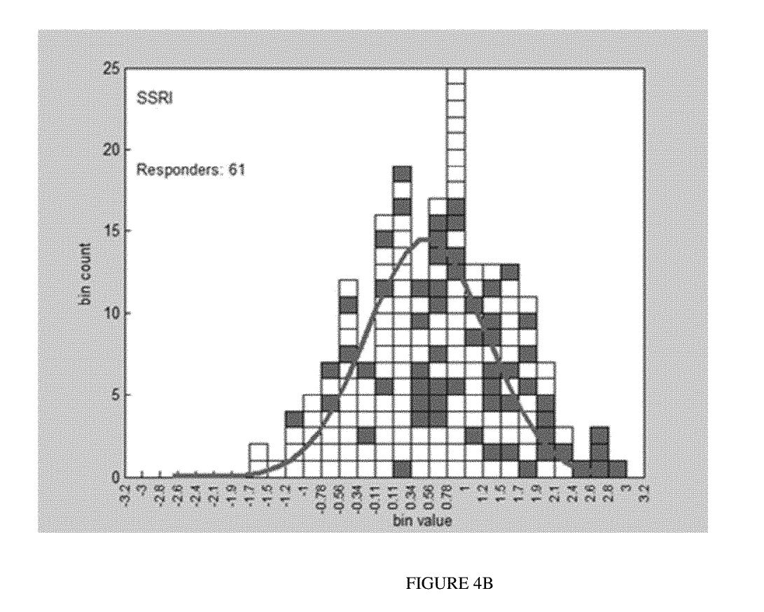

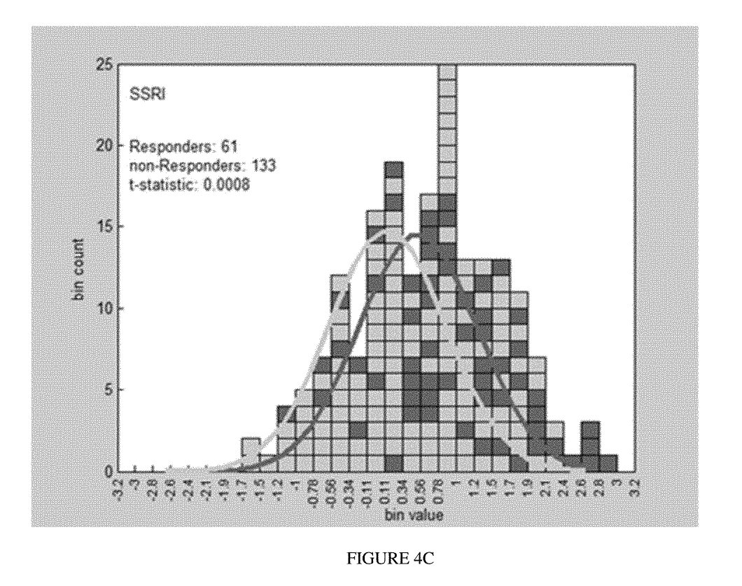

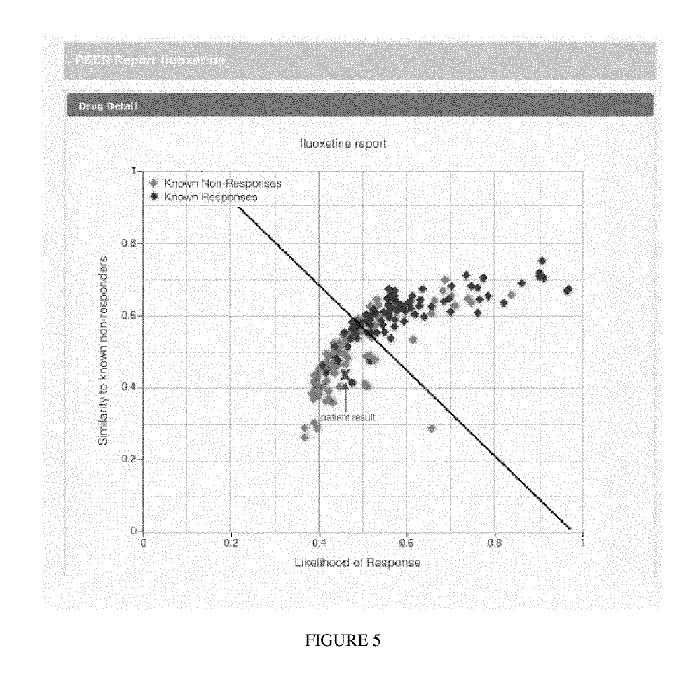

In order to visualize the relationships between patient outcomes and QEEG feature variables the data collected using this process are queried for treatment intervals that meet certain criteria. An example set of criteria might be a treatment interval where a single medication was present and the patient outcome remained stable. In one embodiment, a positive patient outcome remained stable for at least 45 days. In one embodiment, a negative patient outcome remained stable for at least 7 days. The value of an example feature variable from each of the cases in this query can be plotted on histograms. The total treatment interval can be outlined in accordance with compliance with the treatment inclusion criteria. See, FIG. 4A. The cases which represent positive or negative outcomes to treatment can be plotted for analysis. See, FIGS. 4B and 4C. This analysis easily shows that patients with higher values of this particular feature variable (or feature variable pattern) have a greater tendency to respond to the medication in question, SSRI's in this case. Performing a t-test for independent samples comparing medication responders to medication non-responders results in a p-value of <0.05 suggesting that there is a greater than 95% chance that these distributions are different and not due to chance. Note that this particular analysis represents this relationship for only one feature variable (i.e., an QEEG multivariable) that is available from a QEEG analysis. When combined with the PEER database the method produces a report showing a graphic display of the frequency of non-responders to responders of other patients with similar feature variable patterns. See, FIG. 5. In this example, the patient (X) would be predicted to be a non-responder to fluoxetine.

One of skill in the art would know that there are many machine learning techniques available for learning which set of feature variables may be indicative of a group membership (i.e., a responder group or a non-responder group). On such machine learning technique is linear discriminant analysis on the available feature variable set to derive a score that is indicative of the likelihood of any case belonging to one of the groups in the analysis.

C. Model Calibration and Cross Validation

The results of machine learning model development can be rigorously tested against data that has not been used in the development of the model in order to assess the model's full potential. For example, the PEER database methodology can use a standard ten (10)-fold cross validation to test developmental models. In this technique the data are divided in to ten (10) groups, or “folds”. Nine (9) of these folds are used to construct a model and the 10th fold is used to test the model. This process is repeated ten (10) times using a different fold as a test fold each time so that all cases in the dataset have the opportunity to be part of the test dataset. The actual and predicted statistics (e.g., true positive, true negative, false positive and/or false negative) for the test dataset that are reported for the model is the average of those statistics for ten (10) runs.

D. Integrative Analysis Concepts

Using EEG feature variables and/or their patterns differ from a standard quantitative EEG in that it references the quantitative EEG to a normative database and then to a symptomatic database (a database of treated diagnosed patients with a characteristic QEEG feature pattern correlated with a measured therapeutic response). Correlating this data with known historical outcomes may provide treating clinicians with objective physiology-based information with which they can incorporate with their own clinical judgment to make more informed treatment decisions.

One retrospective study examined charts for thirty-three (33) patients with a primary diagnosis of eating disorder and comorbid major depressive disorder or bipolar disorder. Patients underwent a QEEG assessment which provided additional information to the clinicians regarding treatment options. The analysis included twenty-two (22) subjects who accepted treatments based on this information. Subjects whose QEEG data was used for clinical treatment reported significant decreases in associated depressive symptoms (HDRS scores), overall severity of illness (Clinical Global Impression-Severity), and overall clinical global improvement (Clinical Global Impression-Improvement). This cohort also reported fewer inpatient, residential, and partial hospitalization program days following QEEG-directed therapy as compared with the two-year period “trial and error” treatment prior to a QEEG analysis. Greenblatt, et. al.

Another retrospective study reviewed the charts of four hundred and thirty-five (435) patients who elected to undergo QEEG assessment between 2003 through mid-2011 in an outpatient psychiatric clinic. Patients were all non-psychotic psychiatric patients and most were treatment resistant. Two hundred and eighty (280) patients were included in an analysis that demonstrated significant improvement on the Clinical Global Impressions-Improvement (CGI-I) scale from an average score of four (4) at baseline to an average score of one point eighty-five (1.85) at maximum medical improvement (MMI). Quality of Life Questionnaire (QLES-Q) scores improved from an average of forty-seven point one (47.1) at baseline to seventy-one point two (71.2) at MMI. Additionally, improvements were seen on number of medications at MMI, time to reach MMI and number of failed medication trials. (Hoffman)

Another study (Debattista) used a randomized, single-blind, parallel-group, 12-center, US-based study of QEEG-guided pharmacotherapy versus the most effective treatment regimens reported in the NIH-sponsored STAR*D study. Relatively treatment-resistant subjects over the age of eighteen (18) years who failed one or more antidepressants were required to have a QIDS-16-SR score of >13 and a MADRS score of >26 at baseline. All subjects underwent a washout of all current medications (with some protocol-specified exceptions) for at least five half-lives before receiving a report with therapy recommendations following a QEEG analysis. Subjects were then assigned a regimen based on the therapy recommendation report. Control subjects who had failed only SSRI's in their current episode were randomized to receive venlafaxine XR. Control subjects who had failed antidepressants from more than two different classes of antidepressants were randomized to receive a regimen from Steps 2-4 of the STAR*D study. The treatment interval was twelve (12) weeks. The primary outcome measures were change-from-baseline for self-rated QIDS-SR16 and Q-LES-Q-SF. A total of one hundred and fourteen (114) subjects were randomized of which eighty-nine (89) subjects were evaluated. QEEG-guided pharmacotherapy exhibited significantly greater improvement for both primary endpoints, QIDS-SR16 (−6.8 vs. −4.5, p<0.0002) and Q-LES-Q-SF (18.0 vs. 8.9, p<0.0002) as compared to control, respectively, as well as statistical superiority in 9 out of 12 secondary endpoints.

Digital EEG is non-invasive, painless, inexpensive and widely available. The use of the PEER database in numerous studies has improved the frequency of successful patient outcome over controls. When these results are translated to a clinical setting, procedures using the PEER database offer the clinician an easy, objective office procedure that can be used to improve medication selection in patients. PEER does not replace clinician judgment, rather it builds upon it to offer clinicians support for their choices or reasons to utilize caution with others. The field of Psychiatry desperately needs tools like this in order to keep pace with the progress being made in other fields of medicine and to earn the confidence of their patients.

E. PEER Clinical Study Results

One recent study determined whether Psychiatric Electroencephalography Evaluation Registry (PEER) Interactive (an objective, adjunctive tool based on a comparison of a quantitative electroencephalogram to an existing registry of patient outcomes) is more effective than the current standard of care in treatment of subjects suffering from depression. An interim report of an ongoing, 2-year prospective, randomized, double blind, controlled study to evaluate PEER Interactive in guiding medication selection in subjects with a primary diagnosis of depression vs standard treatment was recently published. Iosifescu et al., “The use of the Psychiatric Electroencephalography Evaluation Registry (PEER) to personalize pharmacotherapy” Neuropsychiatric Disease and Treatment 12:2131-2142 (2016). Subjects in treatment at two military hospitals were blinded as to study group assignment and their selfreport symptom ratings were also blinded. Quick Inventory of Depressive Symptomatology, Self-Report (QIDS-SR16) depression scores were the primary efficacy endpoint. One hundred and fifty subjects received a quantitative electroencephalography exam and were randomized to either treatment as usual or PEER-informed pharmacotherapy. Subjects in the control group were treated according to Veterans Administration/Department of Defense Guidelines, the current standard of care. In the experimental group, the attending physician received a PEER report ranking the subject's likely clinical response to on-label medications. In a post hoc interim analysis, subjects were separated into Report Followed and Report Not Followed groups—based on the concordance between their subsequent treatment and PEER medication guidance. The predictive validity of PEER recommendations was evaluated. The results showed a significantly greater improvement in depression scores (QIDS-SR16 P□0.03), reduction in suicidal ideation (Concise Health Risk Tracking Scale-SR7 P□0.002), and post-traumatic stress disorder (PTSD) score improvement (PTSD Checklist Military/Civilian P□0.04) for subjects treated with PEER-recommended medications compared to those who did not follow PEER recommendations.

### VI. Genomic Analysis in Therapy Predictions

A. Metabolic Genomic Analysis

The Cytochrome p450 (also known as CYP450) metabolic pathway comprises a very large family of hemoproteins identified in many living species that act as enzymes to cause oxidative metabolism. p450 enzymes are present in many body tissues, particularly the liver and GI tract and play important roles in hormone synthesis and breakdown, cholesterol synthesis, and vitamin D metabolism. There have been over 7000 distinct cytochrome p450 sequences identified, although there are only about 50 in humans. The hepatic cytochromes are the most widely studied because of their importance to drug metabolism. p450 enzymes may play a role in drug metabolism by facilitating solubility for excretion in the urine or bile. Many drugs affect the activity of p450 via enzyme induction or inhibition. Induction means that a drug stimulates the synthesis of more p450 enzyme to accelerate metabolic capacity. Inhibition means competition between drugs for the enzyme binding site. Two drugs taken together may interact differently with the CYP system and cause changes in CYP-mediated metabolism. The resulting metabolic changes can speed up or slow down drug clearance and contribute to drug-induced side effects or failure of the drug to achieve adequate blood levels. In the worst case, one drug may inhibit CYP-mediated metabolism of another drug leading to drug accumulation and toxicity.

In particular, the p450 enzymes including, but not limited to CYP3A4, CYP2D6, CYP2C19, and CYP1A2 have been associated with psychotropic medications. CYPD26 has genetic polymorphism resulting in marked variation in human metabolic activity. CYP2D6 can be inhibited competitively to affect drug metabolism although it cannot be induced. Approximately 10 percent of Caucasians are poor metabolizers of drugs metabolized by CYP2D6 putting them at some risk for drug accumulation particularly when they take competing drugs. Similarly, 15 to 20 percent of African-Americans and Asian-Americans are poor metabolizers of CYP2C19 compared with only 1 to 5 percent of Caucasians. Several antidepressant and antipsychotic drugs are metabolized by CYP2D6, which means that slow metabolizers given normal dosages may be at some risk for cardiotoxicity, postural hypotension, or oversedation. These drugs include most SSRIs and tricyclics, as well as conventional and atypical antipsychotic medications. Similarly, some SSRIs also inhibit CYP3A enzymes. However, it is important to emphasize that the potency of inhibition varies from drug to drug.

In one recent report, it has been concluded that the p450 system would not be helpful in predicting whether a patient will have a successful outcome with a neuropsychiatric drug. The reference teaches one in the art that while p450 tests show who will be a slow or fast drug metabolizer they do not really tell us which drug will work for which individual patient. Essentially, it is suggested that p450 tests help to assess the risk of side effects and ascertain who might require slower titration or adjusted dosage levels. Thus, fast metabolizers might require higher doses of medication to achieve effective therapeutic levels, and slow metabolizers might require lower doses and slower titration. The assessment concludes that p450 adds minimal clinical value in most cases because: i) the metabolic effects of CYP450 isoenzymes between SSRIs are not substantial; ii) minute differences in p450 metabolism among these drugs may create a false impression that the potential side effect differences between these medications are larger than they really are; and iii) experienced clinicians make drug selection and dosing decisions without p450 testing by deliberate titration. p450 is not a replacement for careful evaluation of the acute symptom profile, previous treatment response, and family history. Nierenberg et al., “Revisiting the Clinical Utility of Cytochrome p450 in Practice” Psychiatry (Edgmont) 4(11):28-30 (2007).

It has been reported that the majority of psychotropic agents are biotransformed by hepatic enzymes:

Substrates, inhibitors, and inducers of major cytochrome isozymes for psychotropic drugs

| Enzyme | Substrate | Inhibitors | Inducers |
|---|---|---|---|
| CYP2D6 | Antipsychotics: Fluphenazine, perphenazine, thioridazine, haloperidol, | Bupropion | None known |
| | chlorpromazine, clozapine, risperidone, olanzapine, aripiprazole, iloperidone, | Duloxetine | |
| | zuclopenthixol | Paroxetine | |
| | Antidepressants: Citalopram, escitalopram, fluoxetine, paroxetine, fluvoxamine, | Fluoxetine | |
| | amitriptyline, nortriptyline, clomipramine, desipramine, imipramine, mirtazapine, | | |
| | venlafaxine | | |
| CYP3A4 | Antipsychotics: Haloperidol, pimozide, clozapine, risperidone, quetiapine, | Nefazodone | Carbamazepine |
| | ziprasidone, aripiprazole, iloperidone, lurasidone | | |
| | Antidepressants: Citalopram, escitalopram, amitriptyline, clomipramine, imipramine, | | |
| | mirtazapine, nefazodone, sertraline, venlafaxine | | |
| | Anxiolytics: Alprazolam, clonazepam, diazepam, buspiron | | |
| | Sedatives/hypnotics: Zolpidem, zaleplon, flurazepam, triazolam | | |
| CYP1A2 | Antipsychotics: Haloperidol, chlorpromazine, perphenazine, thioridazine, clozapine, | Fluvoxamine | Carbamazepine |
| | olanzapine, asenapine, pimozide, loxapine, thiothixene, trifluoperazine | | |
| | Antidepressants: Fluvoxamine, amitriptyline, clomipramine, imipramine, duloxetine, | | |
| | mirtazapine | | |
| CYP2C9 | Valproic acid | Fluoxetine | Carbamazepine |
| | | Fluvoxamine | |
| CYP2C19 | Antipsychotics: Clozapine | Fluvoxamine | Carbamazepine |
| | Antidepressants: Citalopram, escitalopram, clomipramine, imipramine, amitriptyline | | |

Madhusoodanan et al., “A current review of cytochrome P450 interactions of psychotropic drugs” Annals Of Clin Psych 26(2):120-138 (2014).

Roughly 75% of the U.S. population does not metabolize medications normally and genetics can account for 20-95% of the variability in an individual's response to drugs. Villagra et al., “Novel drug metabolism indices for pharmacogenetic functional status based on combinatory genotyping of CYP2C9, CYP2C19 and CYP2D6 genes” Biomarkers In Medicine 5(4):427-438 (2011); and Belle et al., “Genetic Factors in Drug Metabolism” Am Fam Physician. 77(11); 1553-1560 (2008). Furthermore, approximately 2.2 million severe Adverse Drug Events (ADEs) occur in the U.S. every year and: i) are the fourth leading cause of death in the U.S. Lazarou et al., “Incidence of Adverse Drug Reactions in Hospitalized Patients: A Meta-analysis of Prospective Studies” JAMA 279(15):1200-1205 (1998); ii) account for approximately $3.5 billion in extra medical costs annually (U.S. Center for Disease Control and Prevention; Journal of the American Medical Association); iii) three quarters of Medicare-eligible hospitals were fined for ADE-related re-admissions in 2014 (Rau J., “Medicare Fines 2,610 Hospitals In Third Round Of Readmission Penalties” Kaiser Health News kaiserhealthnews.org/news/medicarereadmissions-penalties-2015 (2014); iv) as many as 33% of all potentially clinically significant drug interactions, one of the possible causes of ADEs, are caused by drug-gene and drug-drug-gene interactions and may be missed by drug-drug interaction analysis alone. Verbeurgt et al., “How common are drug and gene interactions? Prevalence in a sample of 1143 patients with CYP2C9, CYP2C19 and CYP2D6 genotyping” Pharmacogenomics 15(5):655-665 (2014).

In response, the US FDA has provided guidelines such that: i) drug-gene interactions should be considered similar in scope to drug-drug interactions; and ii) more than 100 medications known to have drug-gene interactions require FDA warnings on the labels, with recommendation for pharmacogenetic testing prior to use. fda.gov/downloads/Drugs/GuidanceComplianceRegulatoryInformation/Guidances/UCM337169.pdf.

These results are considered to be a direct result of a “trial and error” approach practiced by most current therapeutic strategies. This hit or miss approach leads to drug related problems, such as non-adherence and sub-optimal prescribing and drug administration and diagnosis has been estimated to cost the U.S. as much as $290 billion per year. “Thinking Outside the Pillbox: A System-wide Approach to Improving Patient Medication Adherence for Chronic Disease” A NEHI Research Brief—August 2009: NEHI. For example, cytochrome variants impact more patients than common genetic disorder testing that are relevant for conditions such as breast cancer, cystic fibrosis, Downs syndrome, psychiatric, cardiac, and pain as reported by the Cystic Fibrosis Foundation, BreastCancer.org, National Down Syndrome Society, Administration on Aging, CVS Pharmacy, Wall Street Journal, Medco Health Solutions; and Centers for Disease Control. Furthermore, one-size prescribing can lead to treatment failures and a high cost of care. For example, cancer drugs are ineffective in an average of 75% of patients. www.personalizedmedicinecoalition.org/sites/default/files/files/Case_for_PM_3 rd_edition.pdf.

Recent reviews have demonstrated that each specific medical specialty has problems in predicting patient response under the current administration protocols, for example:
Cardiology—The FDA has included pharmacogenomic information in the labels of 16 cardiology and hematology drugs. Nine of these drugs are processed through the body's highly variable CYP450 pathways. Table of Pharmacogenomic Biomarkers in Drug Labels, FDA, Silver Spring, Md. (2013).
Geriatrics—40% of individuals over 65 take five or more medications and one out of five of these elderly Americans take medications that “may adversely affect coexisting conditions”. Qato et al., “Use of prescription and over-the-counter medications and dietary supplements among older adults in the United States” JAMA 300(24):2867-2878 (2008); and Lorgunpai et al., “Potential therapeutic competition in community-living older adults in the u.s.: use of medications that may adversely affect a coexisting condition” PLoS One 9(2):e89447 (2014).
Pain—Persistent pain impacts 116 million adults and costs the U.S. $560-$635 billion annually; most pain medications (opioids) are metabolized by CYP450 enzymes, thus patients with variations to these genes are at an increased risk of ADEs or treatment failure. Institute of Medicine Committee on Advancing Pain Research, Care, and Education, “Relieving Pain in America: A Blueprint for Transforming Prevention, Care, Education, and Research” Washington, D.C.: National Academies Press; 2011; and Smith H. S., “Opioid Metabolism” Mayo Clin Proc. 84 (7):613-624 (2009).
Psychiatry—Reduced metabolic function is associated with an increased risk of adverse effects in patients taking antidepressants. Ingelman-Sundberg et al., “Influence of cytochrome P450 polymorphisms on drug therapies: pharmacogenetic, pharmacoepigenetic and clinical aspects” Pharmacol Ther. 116:496-526 (2007).

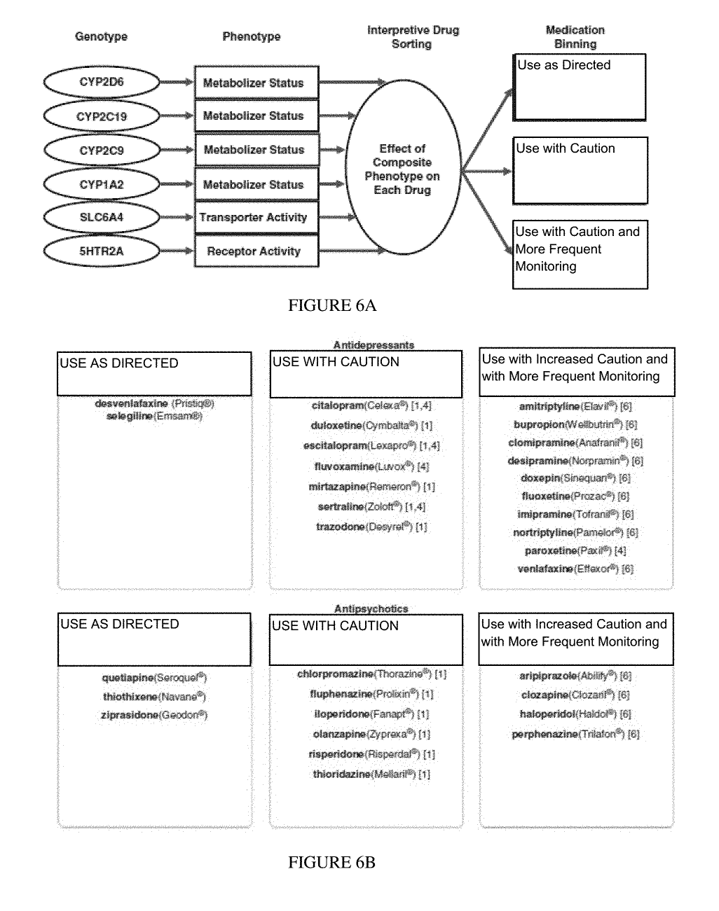

One genomic analysis technique is commercially known as GeneSight®. GeneSight determines a patient's genotype for six specific genes and creates a “composite phenotype” for each drug that is then categories into different risk categories: i) use as directed; ii) use with caution; and iii) use with caution/frequent monitoring. See, FIG. 6A. The report of this genomic analysis is an alphabetical listing of the types of drugs falling into each category. See, FIG. 6B. GeneSight® does not have the capability to rank-order a predicted efficacy between the drugs provided within each risk category based upon the patient's genotype.

Prescribing safe and effective medications is a challenge in psychiatry. While clinical use of pharmacogenomic testing for individual genes has provided some clinical benefit, it has largely failed to show clinical utility. However, pharmacogenomic testing that integrates relevant genetic variation from multiple loci for each medication has shown clinical validity, utility and cost savings in multiple clinical trials. While some challenges remain, the evidence for the clinical utility of “combinatorial pharmacogenomics” is mounting. Expanding education of pharmacogenomic testing is vital to implementation efforts in psychiatric treatment settings with the overall goal of improving medication selection decisions. Benitez et al., “The clinical validity and utility of combinatorial pharmacogenomics: Enhancing patient outcomes” Applied & Translational Genomics 5:47-49 (2015). This report refers to one of the first prospective, open-label trial identified a significant reduction in the GeneSight guided group compared to the standard of care group based on the HAM-D17 as well as the 16 item Clinician Rated Quick Inventory of Depressive Symptomatology (QIDS-C16). This was replicated by a much larger study, which resulted in a significantly improved response on the QIDS-C16 and HAM-D17, as well as the patient reported 9 item Patient Health Questionnaire (PHQ-9), in the GeneSight guided group compared to standard of care. Finally, a smaller placebo-controlled, double-blind study was mentioned that trended towards similar clinical significance showing improvement in the GeneSight group compared to standard of care with double the likelihood of response.

In previous studies, a combinatorial multigene pharmacogenomic test (GeneSight) predicted those patients whose antidepressant treatment for major depressive disorder resulted in poorer efficacy and increased health-care resource utilizations. Another report extended the analysis of clinical validity to these combined data from these studies. Also compared were the outcome predictions of the combinatorial use of allelic variations in genes for four cytochrome P450 (CYP) enzymes (CYP2D6, CYP2C19, CYP2C9 and CYP1A2), the serotonin transporter (SLC6A4) and serotonin 2A receptor (HTR2A) with the outcome predictions for the very same subjects using traditional, single-gene analysis. Depression scores were measured at baseline and 8-10 weeks later for the 119 fully blinded subjects who received treatment as usual (TAU) with antidepressant standard of care, without the benefit of pharmacogenomic medication guidance. For another 96 TAU subjects, health-care utilizations were recorded in a 1-year, retrospective chart review. All subjects were genotyped after the clinical study period, and phenotype subgroups were created among those who had been prescribed a GeneSight panel medication that is a substrate for either CYP enzyme or serotonin effector protein. On the basis of medications prescribed for each subject at baseline, the combinatorial pharmacogenomic (CPGxTIA) GeneSight method categorized each subject into either a green (‘use as directed’), yellow (‘use with caution’) or red category (‘use with increased caution and with more frequent monitoring’) phenotype, whereas the single-gene method categorized the same subjects with the traditional phenotype (for example, poor, intermediate, extensive or ultrarapid CYP metabolizer). The GeneSight combinatorial categorization approach discriminated and predicted poorer outcomes for red category patients prescribed medications metabolized by CYP2D6, CYP2C19 and CYP1A2 (P=0.0034, P=0.04 and P=0.03, respectively), whereas the single-gene phenotypes failed to discriminate patient outcomes. The GeneSight CPGx process also discriminated health-care utilization and disability claims for these same three CYP-defined medication subgroups. The CYP2C19 phenotype was the only single-gene approach to predict health-care outcomes, Multigenic combinatorial testing discriminates and predicts the poorer antidepressant outcomes and greater health-care utilizations by depressed subjects better than do phenotypes derived from single genes. This clinical validity is likely to contribute to the clinical utility reported for combinatorial pharmacogenomic decision support. Altar et al., “Clinical validity: Combinatorial pharmacogenomics predicts antidepressant responses and healthcare utilizations better than single gene phenotypes” Pharmacogenomics J. 15(5):443-451 (2015).

Antidepressants are among the most widely prescribed medications, yet only 35-45% of patients achieve remission following an initial antidepressant trial. The financial burden of treatment failures in direct treatment costs, disability claims, decreased productivity, and missed work may, in part, derive from a mismatch between optimal and actual prescribed medications. One study has reported the results for a one (1) year blinded and retrospective study evaluated at eight direct or indirect health care utilization measures for 96 patients with a DSM-IV-TR diagnosis of depressive or anxiety disorder. The eight measures were evaluated in relation to an interpretive pharmacogenomic test and reporting system, designed to predict antidepressant responses based on DNA variations in cytochrome P450 genes (CYP2D6, CYP2C19, CYP2C9 and CYP1A2), the serotonin transporter gene (SLC6A4) and the serotonin 2A receptor gene (5HTR2A). All subjects had been prescribed at least one of 26 commonly prescribed antidepressant or antipsychotic medications. Subjects whose medication regimen included a medication identified by the gene-based interpretive report as most problematic for that patient and are in the ‘red bin’ (medication status of ‘use with caution and frequent monitoring’), had 69% more total health care visits, 67% more general medical visits, greater than three-fold more medical absence days, and greater than four-fold more disability claims than subjects taking drugs categorized by the report as in the green bin (‘use as directed’) or yellow bin (‘use with caution’). There were no correlations between the number of medications taken and any of the eight healthcare utilization measures. These results demonstrate that retrospective psychiatric pharmacogenomic testing can identify past inappropriate medication selection, which led to increased healthcare utilization and cost. Winner et al., “Psychiatric pharmacogenomics predicts health resource utilization of outpatients with anxiety and depression” Translational Psychiatry 3:e242 (2013).

B. Non-Metabolic Genomic Analysis

In one embodiment, the present invention contemplates a method for identifying non-metabolic drug biomarkers in a patient tissue biopsy. In one embodiment, the non-metabolic drug biomarker is correlated with therapeutic efficacy for treatment of a psychiatric disorder. In one embodiment, the non-metabolic drug biomarker is a blood based biomarker. In one embodiment, the non-metabolic drug biomarker is a cell based biomarker. In one embodiment, the blood based biomarker is detected using an immunoassay. In one embodiment, the cell based biomarker is detected using nucleic acid sequencing and or single nucleotide polymorphisms.

Depression is the leading psychiatric disorder worldwide with a significant economic and emotional strain on society. There is a need for robust biomarkers which will help improve diagnosis and accelerate the drug discovery process. These are objective, peripheral physiological indicators whose presence can be used to predict the probability of onset or presence of depression, stratify according to severity or symptomatology, indicate prognosis and predict or track response to therapeutic interventions. A recently published review addressed several issues pertaining to biomarkers in depression which include transcriptomic, proteomic, genomic and telomeric biomarkers. It is concluded that biomarkers may play a significant role in the psychiatric clinic. Gururajan et al., “Molecular biomarkers of depression” Neurosci Biobehav Rev. 64:101-133 (2016).

Even though one report suggests that biomarker-based research in MDD is still in its relative infancy, two major questions are highlighted: i) what biomarkers reliably distinguish individuals with MDD from those without MDD; and ii) what biomarkers can identify or predict treatment responders versus non-responders? Despite the fact that the lack of independent validation studies now limit the immediate utility of recent positive findings, promising results have emerged pertaining to predictors of antidepressant effectiveness. Uddin M., “Blood-based biomarkers in depression: emerging themes in clinical research” Mol Diagn Ther. 18(5):469-482 (2014).

Investigations of preclinical biomarkers for major depressive disorder (MDD) encompass the quantification of proteins, peptides, mRNAs, or small molecules in blood or urine of animal models. Most studies aim at characterising the animal model by including the assessment of analytes or hormones affected in depressive patients. The ultimate objective is to validate the model to better understand the neurobiological basis of MDD. Stress hormones or inflammation-related analytes associated with MDD are frequently measured. In contrast, other investigators evaluate peripheral analytes in preclinical models to translate the results in clinical settings afterwards. Large-scale, hypothesis-free studies are performed in MDD models to identify candidate biomarkers. Other studies wish to propose new targets for drug discovery. Animal models endowed with predictive validity are investigated, and the assessment of peripheral analytes, such as stress hormones or immune molecules, is comprised to increase the confidence in the target. Finally, since the mechanism of action of antidepressants is incompletely understood, studies investigating molecular alterations associated with antidepressant treatment may include peripheral analyte levels. In conclusion, preclinical biomarker studies aid the identification of new candidate analytes to be tested in clinical trials. They also increase our understanding of MDD pathophysiology and help to identify new pharmacological targets. Carboni L., “Peripheral biomarkers in animal models of major depressive disorder” Dis Markers. 35(1):33-41 (2013).

In order to identify the biological markers for depression, one review publication focused on gathering information on different factors responsible for depression including stress, genetic variations, neurotransmitters, and cytokines and chemokines previously suggested to be involved in the pathophysiology of depression. It is concluded that the biomarkers such as genetic mutations, neurotransmitters, and cytokines can be used for the identification of depressive conditions in the patients. Tamatam et al., “Genetic biomarkers of depression” Indian J Hum Genet. 18(1): 20-33 (2012).

Major depressive disorder (MDD) is common and moderately heritable. Recurrence and early age at onset characterize cases with the greatest familial risk. MDD and the neuroticism personality trait have overlapping genetic susceptibilities. Most genetic studies of MDD have considered a small set of functional polymorphisms relevant to monoaminergic neurotransmission. Meta-analyses suggest small positive associations between the polymorphism in the serotonin transporter promoter region (5-HTTLPR) and bipolar disorder, suicidal behavior, and depression-related personality traits but not yet to MDD itself. This polymorphism might also influence traits related to stress vulnerability. Newer hypotheses of depression neurobiology suggest closer study of genes related to neurotoxic and neuroprotective (neurotrophic) processes and to overactivation of the hypothalamic—pituitary axis, with mixed evidence regarding association of MDD with polymorphisms in one such gene brain-derived neurotrophic factor (BDNF). Levinson D. F., “The genetics of depression: A review” Biol Psychiatry. 60:84-92 (2006).

Recently the C/T single nucleotide polymorphism (SNP) in intron 14 of the DAT1 gene, also referred to as rs40184, has been demonstrated to moderate the effect of perceived maternal rejection on the onset of MDD, as well as on suicidal ideation, thus signifying a gene-by-environment (G×E) interaction in the etiology of MDD. Haeffel et al., “Association between polymorphisms in the dopamine transporter gene and depression: Evidence for a gene-environment interaction in a sample of juvenile detainees” Psychol Sci. 19:62-69 (2008). This particular SNP has also been found to play a genetic role in certain neuropsychiatric and neurological illnesses such as attention deficit hyperactivity disorder, bipolar disorder, and migraine with aura. Mick et al., “Family based association study of pediatric bipolar disorder and the dopamine transporter gene (SLC6A3)” Am J Med Genet B Neuropsychiatr Genet. 147:1182-1185 (2008); and Todt et al., “New genetic evidence for involvement of the dopamine system in migraine with aura” Hum Genet. 125:265-279 (2009).

The norepinephrine transporter (NET), a Na/Cl-dependent substrate specific transporter, terminates noradrenergic signaling by rapid reuptake of neuronally released norepinephrine into presynaptic terminals. NET exerts a fine regulated control over norepinephrine-mediated physiological effects such as depression. As the flanking promoter region of the NET gene, NET T-182C, contains several cis elements that play a role in transcription regulation, changes in this promoter DNA structure may lead to an altered transcriptional activity responsible for a predisposition to MDD. Cohen et al., “The brain derived neurotrophic factor (BDNF) Va166Met polymorphism and recurrent unipolar depression” Am J Med Genet B Neuropsychiatr Genet. 130:37-38 (2004); Kim et al., “A previously undescribed intron and extensive upstream sequence, but not Phox2a-mediated transactivation, are necessary for high level cell type-specific expression of the human norepinephrine transporter gene” J Biol Chem. 274:6507-6518 (1999); and Meyer et al., “Cloning and functional characterization of the human norepinephrine transporter gene promoter” J Neural Transm. 105:1341-1350 (1998). A silent G1287A polymorphism, located at exon 9 of the NET gene, may be a factor in susceptibility to depression. Chang et al., “Lack of association between the norepinephrine transporter gene and major depression in a Han Chinese population” J Psychiatry Neurosci. 32:121-128 (2007).

BDNF is a nerve growth factor that has antidepressant-like effects in animals and may be implicated in the etiology of mood-related phenotypes. However, genetic association studies of the BDNF Va166Met polymorphism (SNP rs6265) in MDD have produced inconsistent results. Meta-analysis of studies compared the frequency of the BDNF Va166Met-coding variant in depressed cases (MDD) and non-depressed controls. MDD is more prevalent in women and in Caucasians and because BDNF allele frequencies differ by ethnicity. BDNF Va166Met polymorphism is of greater importance in the development of MDD in men than in women. Verhagen et al., “Meta-analysis of the BDNF Va166Met polymorphism in major depressive disorder: Effects of gender and ethnicity” Mol Psychiatry. 15:260-271 (2010). In order to further clarify the impact of BDNF gene variation on major depression as well as antidepressant treatment response, association of three BDNF polymorphisms [rs7103411, Va166Met (rs6265) and rs7124442] with major depression and antidepressant treatment response was investigated. Jiang et al., “BDNF variation and mood disorders: A novel functional promoter polymorphism and Va166Met are associated with anxiety but have opposing effects” Neuropsychopharmacol. 30:1353-1361 (2005). All SNPs had main effects on antidepressant treatment response. Results do not support an association between genetic variation in BDNF and antidepressant treatment response or remission. Preliminary studies suggest a potential minor role of genetic variation in BDNF and antidepressant treatment outcome in the context of melancholic depression. Domschke et al., “Brain-derived neurotrophic factor (BDNF) gene: No major impact on antidepressant treatment response” Int J Neuropsychopharmacol. 13:93-101 (2010). Identification of genetic polymorphisms in the BDNF gene and assessment of their frequencies and associations with MDD or antidepressant response have recently been reported. For example, single-nucleotide polymorphisms (SNPs), untranslated regions, in coding sequences, in introns, and upstream regions; 3 of 4 rare coding SNPs were observed to be non synonymous. Association analyses of patients with MDD and controls showed that 6 SNPs were associated with MDD (rs12273539, rs11030103, rs6265, rs28722151, rs41282918, and rs11030101) and two haplotypes in different blocks (one including Va166, another near exon VIIIh) were significantly associated with MDD. One recently reported 5′ untranslated region SNP, rs61888800, was associated with antidepressant response after adjusting for age, sex, medication, and baseline score on the 21-item Hamilton Depression Rating Scale. Licinio et al., “Novel sequence variations in the brain-derived neurotrophic factor gene and association with major depression and antidepressant treatment response” Arch Gen Psychiatry. 66:488-497 (2009). Alterations in BDNF-signaling pathways may play a role in the pathophysiology of MDD. Five SNPs in three BDNF signal-transduction pathway genes (BDNF, GSK3B, and AKT1) were used as genetic biomarkers of depression. An allelic association between the GSK3B SNP rs6782799 and MDD was found in these samples. Further gene-gene interaction analyses showed a significant effect of a two-locus BDNF/GSK3B interaction with MDD (GSK3B rs6782799 and BDNF rs7124442) and also for a three-locus interaction (GSK3B rs6782799, BDNF rs6265, and BDNF rs7124442). These findings support the assertion that the GSK3B gene is an important susceptibility factor for MDD in a Han Chinese population. Zhang et al., “Genetic association of the interaction between the BDNF and GSK3B genes and major depressive disorder in a Chinese population” J Neural Transm. 117:393-401 (2010).

The 5-HTT gene regulates brain serotonin neurotransmission by removing the neurotransmitter from the extracellular space. Since the development of the selective serotonin reuptake-inhibitors, a putative role for 5-HTT in the etiology of depression has been explored. The discovery of a functional 5-HTT polymorphism has provided a novel tool to further scrutinize the role of serotonergic neurons in depression. A repeat of 20-23 base pairs has been observed as a motif within a polymorphic region of the 5-HTT gene and it occurs as two prevalent alleles: one consisting of 14 repeats (S allele) and another of 16 repeats (L allele). This functional polymorphism in the promoter region, termed 5-HTTLPR, alters transcription of the serotonin transporter gene. The S allele leads to less transcriptional efficiency of serotonin and it can partly account for anxiety-related personality traits. Heils et al., “The human serotonin transporter gene polymorphism-basic research and clinical implications” J Neural Transm. 104:1005-1014 (1997); and Heils et al., “Allelic variation of human serotonin transporter gene expression” J Neurochem. 1996; 66:2621-2624 (1996). Two serotonin 2A receptor (HTR2A) SNPs recently reported to be associated with antidepressant treatment response in STARD (rs7997012; rs1928040) for association with treatment response in two independent Caucasian samples of patients with a Major Depressive Episode. SNP rs7997012 was significantly associated with remission after 5 weeks providing first replicative support for the initial finding, with however, an inverse allelic association as compared to the STAR*D sample. Lucae et al., “HTR2A gene variation is involved in antidepressant treatment response” Eur Neuropsychopharmacol. 20:65-68 (2010). Another common polymorphism is a variable number tandem repeat (VNTR) in intron 2 (STin2), which has three alleles consisting of either 9 (STin2.9), 10 (STin2.10), or 12 (STin2.12) repeats, were shown to be in linkage disequilibrium, with the positive association between the STin2 allele 10 and the 5-HTTLPR L allele. Collier et al., “A novel functional polymorphism within the promoter of the serotonin transporter gene: possible role in susceptibility to affective disorders” Mol Psychiatry. 1:453-460 (1996). Variation at the VNTR can also influence expression of the transporter with the polymorphic VNTR regions acting as transcriptional regulators although it is likely to have no significant effect on function. McKenzie et al., “A serotonin transporter gene intron 2 polymorphic region, correlated with affective disorders, has allele-dependent differential enhancer-like properties in the mouse embryo” Proc Natl Acad Sci USA. 96:15251-15255 (1999).

A role of inflammation in depression long been suspected. Smith R. S., “The macrophage theory of depression” Med Hypotheses 35:298-306 (1991). Since then, several studies have reported a link between MDD, or depressive symptoms, and a variety of inflammatory and immune biomarkers. Empana et al., “Contributions of depressive mood and circulating inflammatory markers to coronary heart disease in healthy European men: The Prospective Epidemiological Study of Myocardial Infarction (PRIME) Circulation” 111:2299-2305 (2005); Kling et al., “Sustained low-grade pro-inflammatory state in unmedicated, remitted women with major depressive disorder as evidenced by elevated serum levels of the acute phase proteins C-reactive protein and serum amyloid A” Biol Psychiatry. 62:309-313 (2007); and Miller et al., “Clinical depression and inflammatory risk markers for coronary heart disease” Am J Cardiol. 90:1279-1283 (2002).

Depression may cause inflammation through altered neuroendocrine function and central adiposity. Carney et al., “Depression as a risk factor for cardiac mortality and morbidity: A review of potential mechanisms” J Psychosom Res. 53:897-902 (2002). However, depression may also be a consequence of inflammation, since a pathogenic role of inflammatory cytokines in the etiology of depression has been described. Raison et al., “Cytokines sing the blues: inflammation and the pathogenesis of depression” Trends Immunol. 27:24-31 (2006). Although given less consideration, a third possibility is that depression is a marker of some other underlying dimension that is separately linked to depression and inflammation. Recently, it has been proposed that such underlying factor could be a specific genetic makeup. McCaffery et al., “Common genetic vulnerability to depressive symptoms and coronary artery disease: A review and development of candidate genes related to inflammation and serotonin” Psychosom Med. 68:187-200 (2006).

MPO is an enzyme of the innate immune system, which exhibits a wide array of proatherogenic features. McMillen et al., “Expression of human myeloperoxidase by macrophages promotes atherosclerosis in mice” Circulation 111:2798-2804 (2005). MPO is secreted upon leukocyte activation, contributing to innate host defenses. However, it also increases oxidative stress, thereby contributing to tissue damage during inflammation and atherogenesis. For example, elevated levels of antioxidant enzymes, particularly superoxide dismutase (SOD) and biomarkers of oxidation, such as malondialdehyde, were found in plasma, red blood cells, or other peripheral tissues of acutely depressed MDD patients compared with controls. In some cases, but not others, these abnormalities were reduced with antidepressant treatment. Bilici et al., “Antioxidative enzyme activities and lipid peroxidation in major depression: Alterations by antidepressant treatments” J Affect Disord. 64:43-51 (2001); and Sarandol et al., “Major depressive disorder is accompanied with oxidative stress: Short-term antidepressant treatment does not alter oxidative/antioxidative systems” Hum Psychopharmacol. 22:67-73 (2007). SOD coenzyme concentrations are also higher in postmortem brain tissue (prefrontal cortex) of MDD patients than in control brains. Michel et al., “Evidence for oxidative stress in the frontal cortex in patients with recurrent depressive disorder A postmortem study” Psychiatry Res. 151:145-150 (2007).

### V. Combinatorial QEEG and Genomic Analysis

Machine learning applications have demonstrated superior predictive accuracy in other areas of medicine which have eluded traditional, expert-driven solutions. In particular, machine learning algorithms trained by real-world data have demonstrated results in clinical medicine that exceed those of experts in neuroimaging, cytology, and other diagnostics. In some embodiments, the present invention contemplates machine learning to identify predictive features from individual electrophysiology and pharmacogenomic findings, referenced to a large clinical database of longitudinal outcomes (e.g., for example, approximately 10,400 patients), In one embodiment, the present invention contemplates “digital phenotyping” comprising a combination of algorithms that provides a significantly greater accuracy and actionable findings than currently found in a routine clinical practice setting,

The present invention provides methods to solve a contemporary problem in the field of psychiatric treatment that has been summarized as “More Medications≠Better Outcomes, Centers for Disease Control, MS Health 20161 (April 2016). For example, while more people are getting more of today's treatment it is not clear, on a population basis, that the outcomes are any better. As discussed herein, some studies may show effectiveness of a therapy on a population, individual response is highly variable, causing those in the art to conclude that “It is still very much trial and error”. Insel et al., “NIMH Efforts Seek Personalized Medicine Approaches to Prevent Brain Disorders” AJMC (2014). For example, an evaluation by the Food and Drug Administration found that only 51% of psychiatric drug trials had a positive outcome, which differed considerably from the conventional opinion of those in the art that 94% of the published trials had a positive outcome. Turner et al, “Selective Publication of Antidepressant Trials and its Influence on Apparent Efficacy” NEJM (2008). In response, PEER was developed in the 1990s by a group of California physicians, who wanted objective information to improve quality of care. The online registry captures realworld outcomes for specific medications referenced to a standard, reliable measure of electrophysiology: EEG, After uploading an EEG, PEER is delivered to physicians as a web-based medication response report, much like an antibiotic assay. The Registry currently represents n=10,400 unique patients, 38,000 outcome correlations with EEG findings.

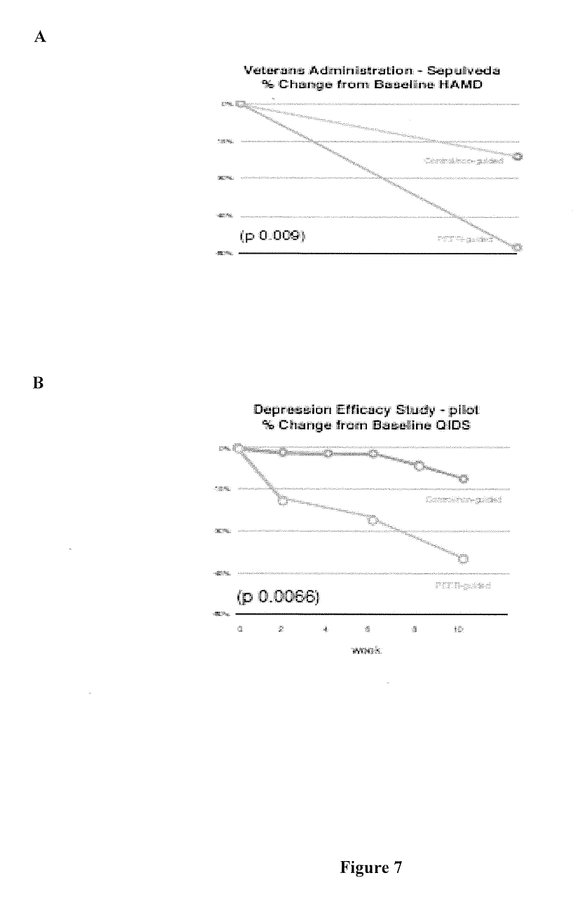

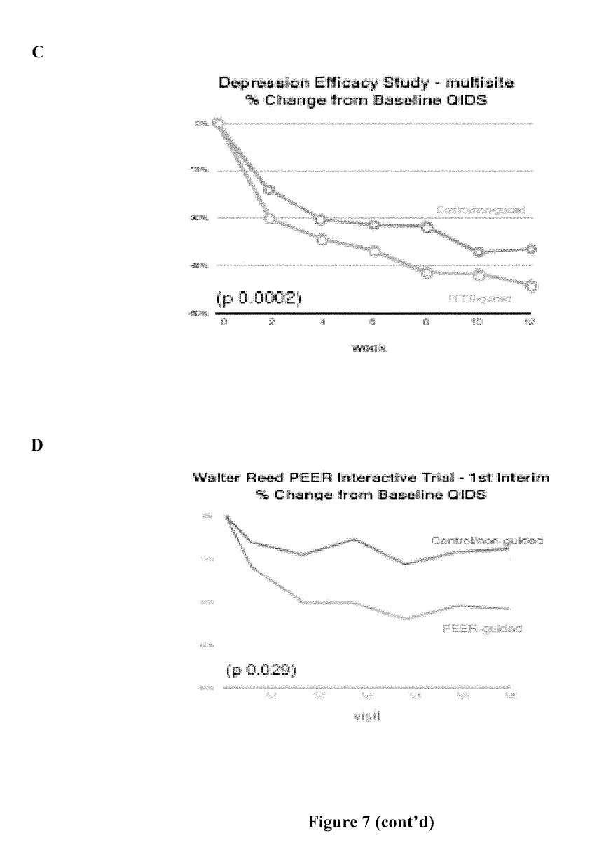

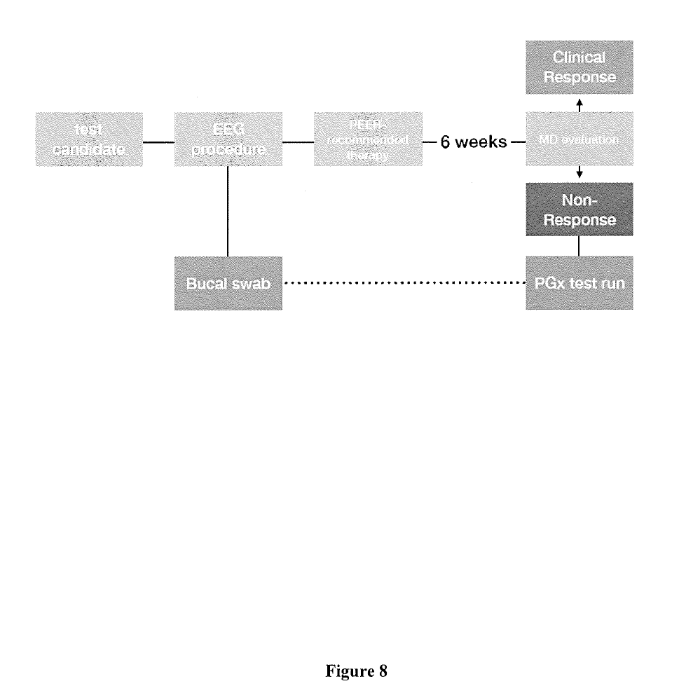

In several randomized, double-blinded controlled trials of PEER and predecessor rEEG studies, as discussed below, PEER guidance was compared to Treatment As Usual (TAU) hi the treatment of patients with Treatment-Resistant Depression (MD). See, FIG. 7 A-D. The data from four of these studies (e.g., Veterans Administration—Sepulveda (J Am Physicians & Surgeons, 2007), FIG. 7A; Depression Efficacy Pilot Study 12 (NCDEU, 2009), FIG. 7B; Depression Efficacy Study—Harvard/Stanford multi-site (1 Psych Res, 2011), FIG. 7C; and Walter Reed PEER interactive Trial—(Neuropsychiatric Disease and Treatment, 2016), See, FIG. 7D. Overall, these studies showed that following the use of PEER medication efficacy tripled where the mean change from baseline was −47% when guided by PEER as opposed to −16% under conventional “trial and error” prescription strategies. Over 50% improvement was observed in treatment efficiency where improvements in those subjects following the PEER therapies surpassed control groups in less than half the clinical visits. Schiller et al., “Disrupting Trial & Error: Can “Big Data” Help Physicians Improve Quality of Treatment?” Military Health Services Research Symposium, Poster, (2016).

A. Co-Variate Analysis of QEEG and Genomic Factors

It has been reported that QEEG measures, Oddball evoked potential responses (ERPs), genotypes and neuropsychological testing scores all have differential predictive power in assessing antidepressant therapy efficacy. Each category was individually assessed against changes in HAM-D scores to determine relative predictive power before and after antidepressant treatment by means of scatterplots and correlation analysis. The individual contribution of each type of predictor was determined by entry of the respective data into one of several statistical linear regression models. For example, in depression therapy relative predictive power was compared between genotypes of catechol-O-methyltransferase (COMT), QEEG measurements and cognitive testing results. In particular, the Met/Met COMT genotype best predicted overall drug efficacy, while the neuropsychological test results for verbal memory performance best predicted cognitive performance, and a QEEG measurement of frontal theta power as measured from the Fz electrode was the best predictor of changes in HAM-D test scores. Spronk et al., “An investigation of EEG, genetic and cognitive markers of treatment response to antidepressant medication in patients with major depressive disorder: A pilot study” J Affect Disord 128:41-48 (2011). The QEEG measurements in this study were limited to Fast Fourier transformation of raw EEG measurements into frequency power spectra (e.g., Alpha, Beta, Theta and Delta) that were square-root-transformed to approximate the normal distributional assumptions required by parametric statistical methods. These studies did not further convert any QEEG data into specific multivariate determinations reflecting activity at specific brain regions. While two genes were tested, COMT and brain derived neurotrophic factor (BDNF), only the data from COMT was evaluated and considered for potential predictive power. Despite the above results when each predictor was evaluated independently, the results were different when an integrative statistical model compared the simultaneous relative contribution of all four predictors to the variance of HAM-D score improvement. The integrative model showed that Oddball ERP (N1 amplitude at the Pz electrode) and total memory score from the cognitive testing accounted for the majority of the variance. Notably, this means that the contribution of QEEG measure and/or the genotypic measure either individually, or in combination, was not significant when compared with the contributions of Oddball ERP and total memory score. The study concluded that these results show that the utility of combining measures from different domains (integrative model: R²=60.2%) showed almost no overlap (roughly 3% of variance) between the individual comprising predictors—as the sum of variances explained in the separate models (independent models: R²=63.2%.). In conclusion, this study finds no predictive power in antidepressant efficacy when QEEG measures are combined with genotype measures, even though each may have some predictive power when individually evaluated on specific recovery parameters.

B. Machine-Learning Applications for Drug Efficacy Analysis

In one study, a machine-learning algorithm was applied to the problem of determining if low antidepressant treatment efficacy might be improved by matching patients to interventions. At present, clinicians have no empirically validated mechanisms to assess whether a patient with depression will respond to a specific antidepressant. An algorithm was developed to assess whether patients would achieve symptomatic remission from a 12-week course of citalopram. Patient-reported depression data was collected (n=4041, with 1949 completers) from level 1 of the Sequenced Treatment Alternatives to Relieve Depression (STAR*D; ClinicalTrials.gov, number NCT00021528) to identify variables that were most predictive of treatment outcome, and used these variables to train a machine-learning model to predict clinical remission. The model was externally validated with an escitalopram treatment group (n=151) from an independent clinical trial (Combining Medications to Enhance Depression Outcomes [COMED]; ClinicalTrials.gov, number NCT00590863). The data identified 25 variables that were most predictive of treatment outcome from 164 patient-reportable variables, and used these to train the model. The model was internally cross-validated, and predicted outcomes in the STAR*D cohort with accuracy significantly above chance (64.6% [SD 3.2]; p<0.0001). The model was externally validated in the escitalopram treatment group (N=151) of COMED (accuracy 59.6%, p=0.043). The model also performed significantly above chance in a combined escitalopram-buproprion treatment group in COMED (n=134; accuracy 59.7%, p=0.023), but not in a combined venlafaxine-mirtazapine group (n=140; accuracy 51.4%, p=0.53), suggesting specificity of the model to underlying mechanisms. This suggests that by building statistical models by mining existing clinical trial data prospective identification of patients can be made who are likely to respond to a specific antidepressant. Chekroud et al., “Cross-trial prediction of treatment outcome in depression: a machine learning approach” Lancet Psychiatry 3(3):243-250 (2016).

C. QEEG Feature Variable/Single Gene Combinations

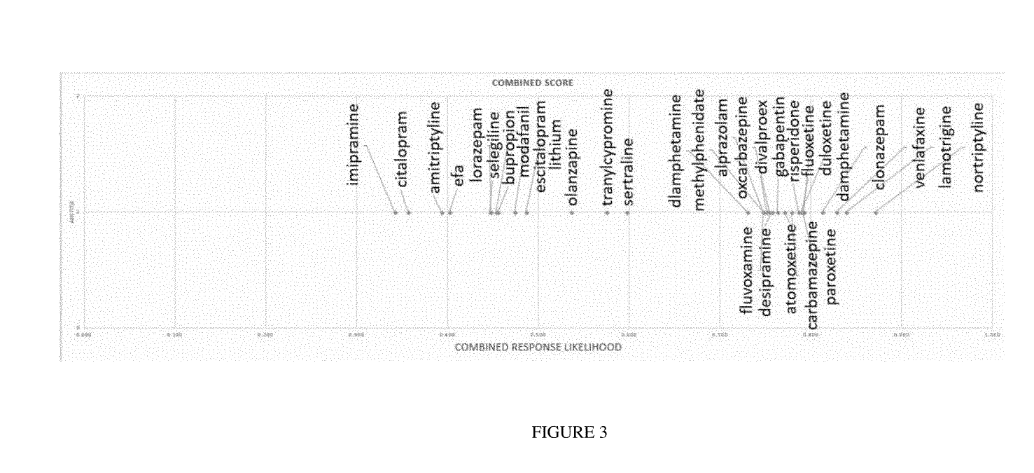

In one embodiment, the present invention contemplates a method for predicting psychiatric drug efficacy using a combination of patient data comprising at least one QEEG feature variable and single gene genotype. See, FIG. 3. The combined data analysis for the QEEG feature variable and the single gene genotype computes a combination metric displayed on the x-axis of FIG. 3, and takes the following form:

M=\sqrt{(G−Gₒ)²+(P−Pₒ)²}/\sqrt{2}

where:

M=the value of the metric for the drug on a scale of 0 to 1

G=the score for the drug on the genetic metabolic panel

Gₒ=an arbitrary origin for genetic test scores

P=the score for the drug on the PEER report

Pₒ=and arbitrary origin for PEER report scores

In one embodiment, the present invention contemplates a QEEG feature variable comprising at least three component EEG terms See, Table 1.

TABLE 1
QEEG Feature Variables: Component Term Defintions

| First term | Second term | Third term |
|---|---|---|
| abs_power = absolute power | ant = anterior | delta = 1.0-3.5 hz |
| rel_power = relative power | post = posterior | theta = 4.0-7.5 hz |
| asymmetry = asymmetry | llat = left lateral | alpha = 8.0-12.0 Hz |
| burst_amp = burst amplitude | rlat = right lateral | beta = 12.5-25.0 Hz |
| burst_dur = burst duration | intlat = intra | alpha1 = 8.8-10.0 Hz |
| burst_int = burst interval | lateral | alpha2 = 10.0-12.0 Hz |
| burst_per_sec = bursts per | | beta1 = 12.0-15.0 Hz |
| second | | beta2 = 15.0-17.5 Hz |
| peak_freq = peak frequency | | beta3 = 18.0-25.0 Hz |
| phase_lag = phase lag | | high beta = 25.5-30.0 |
| power_ratio = power ratio | | Hz |
| abs_power = absolute power | | |
| rel_power = relative power | | |
| asymmetry = asymmetry | | |
| burst_amp = burst amplitude | | |
| burst_dur = burst duration | | |
| burst_int = burst interval | | |
| burst_per_sec = bursts per | | |
| second | | |
| peak_freq = peak frequency | | |
| phase_lag = phase lag | | |
| power_ratio = power ratio | | |

In one embodiment, the present invention contemplates a plurality of QEEG feature variables (e.g., multivariables) comprising at least three component terms. In one embodiment, the QEEG feature variable is a combination derived from some of the 7,200 or so univariate QEEG data points. In some embodiments, the QEEG feature variables contemplated herein include, but are not limited to, those listed below. See, Table 2.

TABLE 2
Representative QEEG Feature Variables

| | |
|---|---|
| 1 | abs_power_ant_delta |
| 2 | abs_power_ant_theta |
| 3 | abs_power_ant_alpha1 |
| 4 | abs_power_ant_alpha2 |
| 5 | abs_power_ant_beta1 |
| 6 | abs_power_ant_beta2 |
| 7 | abs_power_ant_beta3 |
| 8 | abs_power_post_delta |
| 9 | abs_power_post_theta |
| 10 | abs_power_post_alpha1 |
| 11 | abs_power_post_alpha2 |
| 12 | abs_power_post_beta1 |
| 13 | abs_power_post_beta2 |
| 14 | abs_power_post_beta3 |
| 15 | asymmetry_ant_delta |
| 16 | asymmetry_ant_theta |
| 17 | asymmetry_ant_alpha1 |
| 18 | asymmetry_ant_alpha2 |
| 19 | asymmetry_ant_beta1 |
| 20 | asymmetry_ant_beta2 |
| 21 | asymmetry_ant_beta3 |
| 22 | asymmetry_post_delta |
| 23 | asymmetry_post_theta |
| 24 | asymmetry_post_alpha1 |
| 25 | asymmetry_post_alpha2 |
| 26 | asymmetry_post_beta1 |
| 27 | asymmetry_post_beta2 |
| 28 | asymmetry_post_beta3 |
| 29 | asymmetry_llat_delta |
| 30 | asymmetry_llat_theta |
| 31 | asymmetry_llat_alpha1 |
| 32 | asymmetry_llat_alpha2 |
| 33 | asymmetry_llat_beta1 |
| 34 | asymmetry_llat_beta2 |
| 35 | asymmetry_llat_beta3 |
| 36 | asymmetry_rlat_delta |
| 37 | asymmetry_rlat_theta |
| 38 | asymmetry_rlat_alpha1 |
| 39 | asymmetry_rlat_alpha2 |
| 40 | asymmetry_rlat_beta1 |
| 41 | asymmetry_rlat_beta2 |
| 42 | asymmetry_rlat_beta3 |
| 43 | asymmetry_intlat_delta |
| 44 | asymmetry_intlat_theta |
| 45 | asymmetry_intlat_alpha1 |
| 46 | asymmetry_intlat_alpha2 |
| 47 | asymmetry_intlat_beta1 |
| 48 | asymmetry_intlat_beta2 |
| 49 | asymmetry_intlat_beta3 |
| 50 | burst_amp_ant_delta |
| 51 | burst_amp_ant_theta |
| 52 | burst_amp_ant_alpha1 |
| 53 | burst_amp_ant_alpha2 |
| 54 | burst_amp_ant_beta1 |
| 55 | burst_amp_ant_beta2 |
| 56 | burst_amp_ant_beta3 |
| 57 | burst_amp_post_delta |
| 58 | burst_amp_post_theta |
| 59 | burst_amp_post_alpha1 |
| 60 | burst_amp_post_alpha2 |
| 61 | burst_amp_post_beta1 |
| 62 | burst_amp_post_beta2 |
| 63 | burst_amp_post_beta3 |
| 64 | burst_dur_ant_delta |
| 65 | burst_dur_ant_theta |
| 66 | burst_dur_ant_alpha1 |
| 67 | burst_dur_ant_alpha2 |
| 68 | burst_dur_ant_beta1 |
| 69 | burst_dur_ant_beta2 |
| 70 | burst_dur_ant_beta3 |
| 71 | burst_dur_post_delta |
| 72 | burst_dur_post_theta |
| 73 | burst_dur_post_alpha1 |
| 74 | burst_dur_post_alpha2 |
| 75 | burst_dur_post_beta1 |
| 76 | burst_dur_post_beta2 |
| 77 | burst_dur_post_beta3 |
| 78 | burst_int_ant_delta |
| 79 | burst_int_ant_theta |
| 80 | burst_int_ant_alpha1 |
| 81 | burst_int_ant_alpha2 |
| 82 | burst_int_ant_beta1 |
| 83 | burst_int_ant_beta2 |
| 84 | burst_int_ant_beta3 |
| 85 | burst_int_post_delta |
| 86 | burst_int_post_theta |
| 87 | burst_int_post_alpha1 |
| 88 | burst_int_post_alpha2 |
| 89 | burst_int_post_beta1 |
| 90 | burst_int_post_beta2 |
| 91 | burst_int_post_beta3 |
| 92 | bursts_per_sec_ant_delta |
| 93 | bursts_per_sec_ant_theta |
| 94 | bursts_per_sec_ant_alpha1 |
| 95 | bursts_per_sec_ant_alpha2 |
| 96 | bursts_per_sec_ant_beta1 |
| 97 | bursts_per_sec_ant_beta2 |
| 98 | bursts_per_sec_ant_beta3 |
| 99 | bursts_per_sec_post_delta |
| 100 | bursts_per_sec_post_theta |
| 101 | bursts_per_sec_post_alpha1 |
| 102 | bursts_per_sec_post_alpha2 |
| 103 | bursts_per_sec_post_beta1 |
| 104 | bursts_per_sec_post_beta2 |
| 105 | bursts_per_sec_post_beta3 |
| 106 | coherence_ant_delta |
| 107 | coherence_ant_theta |
| 108 | coherence_ant_alpha1 |
| 109 | coherence_ant_alpha2 |
| 110 | coherence_ant_beta1 |
| 111 | coherence_ant_beta2 |
| 112 | coherence_ant_beta3 |
| 113 | coherence_post_delta |
| 114 | coherence_post_theta |
| 115 | coherence_post_alpha1 |
| 116 | coherence_post_alpha2 |
| 117 | coherence_post_beta1 |
| 118 | coherence_post_beta2 |
| 119 | coherence_post_beta3 |
| 120 | coherence_llat_delta |
| 121 | coherence_llat_theta |
| 122 | coherence_llat_alpha1 |
| 123 | coherence_llat_alpha2 |
| 124 | coherence_llat_beta1 |
| 125 | coherence_llat_beta2 |
| 126 | coherence_llat_beta3 |
| 127 | coherence_rlat_delta |
| 128 | coherence_rlat_theta |
| 129 | coherence_rlat_alpha1 |
| 130 | coherence_rlat_alpha2 |
| 131 | coherence_rlat_beta1 |
| 132 | coherence_rlat_beta2 |
| 133 | coherence_rlat_beta3 |
| 134 | coherence_intlat_delta |
| 135 | coherence_intlat_theta |
| 136 | coherence_intlat_alpha1 |
| 137 | coherence_intlat_alpha2 |
| 138 | coherence_intlat_beta1 |
| 139 | coherence_intlat_beta2 |
| 140 | coherence_intlat_beta3 |
| 141 | peak_freq_ant_delta |
| 142 | peak_freq_ant_theta |
| 143 | peak_freq_ant_alpha1 |
| 144 | peak_freq_ant_alpha2 |
| 145 | peak_freq_ant_beta1 |
| 146 | peak_freq_ant_beta2 |
| 147 | peak_freq_ant_beta3 |
| 148 | peak_freq_post_delta |
| 149 | peak_freq_post_theta |
| 150 | peak_freq_post_alpha1 |
| 151 | peak_freq_post_alpha2 |
| 152 | peak_freq_post_beta1 |
| 153 | peak_freq_post_beta2 |
| 154 | peak_freq_post_beta3 |
| 155 | phase_lag_ant_delta |
| 156 | phase_lag_ant_theta |
| 157 | phase_lag_ant_alpha1 |
| 158 | phase_lag_ant_alpha2 |
| 159 | phase_lag_ant_beta1 |
| 160 | phase_lag_ant_beta2 |
| 161 | phase_lag_ant_beta3 |
| 162 | phase_lag_post_delta |
| 163 | phase_lag_post_theta |
| 164 | phase_lag_post_alpha1 |
| 165 | phase_lag_post_alpha2 |
| 166 | phase_lag_post_beta1 |
| 167 | phase_lag_post_beta2 |
| 168 | phase_lag_post_beta3 |
| 169 | phase_lag_llat_delta |
| 170 | phase_lag_llat_theta |
| 171 | phase_lag_llat_alpha1 |
| 172 | phase_lag_llat_alpha2 |
| 173 | phase_lag_llat_beta1 |
| 174 | phase_lag_llat_beta2 |
| 175 | phase_lag_llat_beta3 |
| 176 | phase_lag_rlat_delta |
| 177 | phase_lag_rlat_theta |
| 178 | phase_lag_rlat_alpha1 |
| 179 | phase_lag_rlat_alpha2 |
| 180 | phase_lag_rlat_beta1 |
| 181 | phase_lag_rlat_beta2 |
| 182 | phase_lag_rlat_beta3 |
| 183 | phase_lag_intlat_delta |
| 184 | phase_lag_intlat_theta |
| 185 | phase_lag_intlat_alpha1 |
| 186 | phase_lag_intlat_alpha2 |
| 187 | phase_lag_intlat_beta1 |
| 188 | phase_lag_intlat_beta2 |
| 189 | phase_lag_intlat_beta3 |
| 190 | power_ratio_ant_delta_theta |
| 191 | power_ratio_ant_delta_alpha |
| 192 | power_ratio_ant_delta_beta |
| 193 | power_ratio_ant_delta_high_beta |
| 194 | power_ratio_ant_theta_alpha |
| 195 | power_ratio_ant_theta_beta |
| 196 | power_ratio_ant_theta_high_beta |
| 197 | power_ratio_ant_alpha_beta |
| 198 | power_ratio_ant_alpha_high_beta |
| 199 | power_ratio_ant_beta_high_beta |
| 200 | power_ratio_post_delta_theta |
| 201 | power_ratio_post_delta_alpha |
| 202 | power_ratio_post_delta_beta |
| 203 | power_ratio_post_delta_high_beta |
| 204 | power_ratio_post_theta_alpha |
| 205 | power_ratio_post_theta_beta |
| 206 | power_ratio_post_theta_high_beta |
| 207 | power_ratio_post_alpha_beta |
| 208 | power_ratio_post_alpha_high_beta |
| 209 | power_ratio_post_beta_high_beta |
| 210 | rel_power_ant_delta |
| 211 | rel_power_ant_theta |
| 212 | rel_power_ant_alpha1 |
| 213 | rel_power_ant_alpha2 |
| 214 | rel_power_ant_beta1 |
| 215 | rel_power_ant_beta2 |
| 216 | rel_power_ant_beta3 |
| 217 | rel_power_post_delta |
| 218 | rel_power_post_theta |
| 219 | rel_power_post_alpha1 |
| 220 | rel_power_post_alpha2 |
| 221 | rel_power_post_beta1 |
| 222 | rel_power_post_beta2 |
| 223 | rel_power_post_beta3 |

In one embodiment, the present invention contemplates a method using machine learning, for extracting QEEG feature variables which correlate to patient response/non-response to individual medications and medication classes that are compiled in a large clinical outcome registry (e.g., a database) for patients with known mental disorders and documented therapy outcomes. As listed above, these QEEG feature variables are based on up to 7,200 individual univariate variables derived from a standard QEEG evaluating which provide measurements including, but not limited to, frequency, power, coherence, symmetry, phase, etc. of an individual's baseline EEG.

In one embodiment, the method further comprises selecting the QEEG feature variables using a machine learning algorithm. Although it is not necessary to understand the mechanism of an invention, the present method represents a significant improvement over current methods used to personalize pharmacotherapy. Historically, others have used individual QEEG variables or single gene pharmacogenomic assays to attempt to predict medication response. However, these prior evaluations have encountered the following disadvantages:
1. QEEG efforts have yielded findings limited to only a few medications, or only a few expert-derived features (RACC), resulting in relatively low predictiveness in real world clinical settings. (cf: Arns et al. 2016, supra).
2. Evidence for pharmacogenomic findings has been limited to drug metabolism, CYP450 enzyme-related poor and/or rapid metabolism, which is found in only about 15% of the population.
   In contrast, the presently contemplated invention overcomes these disadvantages by developing classifiers of response/non-response by using:
1. The largest existing dataset of longitudinal clinical outcomes (PEER—Psychiatric EEG Evaluation Registry, n=10,400 unique patients with 38,000 outcomes over multiple medication intervals). The unique contribution of machine learning to prediction of phenotypic response to individual medications is at the core of our product; since most drug trials involve small samples (100-200 patients receive active medications) with NO active brain measure, there is little information available to differentiate an individual patient's response to any one medication. But with large datasets of known responders and non-responders to individual medications, using machine learning, we are able to identify specific characteristics of the human EEG which predict response.
2. Machine learning that continues to improve predictive accuracy as additional outcomes are added to the database.
3. No a priori hypotheses—pure Machine Learning (cf Wade et al., 2016, supra). Instead, the present invention uses improved multiple classifiers per drug/drug class, as opposed to the single classifiers used in current research.
4. Pharmacogenomics to narrow the range (increase predictiveness) of brain-based predictors.
5. Integrated, combinatorial algorithms are used to generate a single # outcome prediction/score for each medication and medication class.
   Although it is not necessary to understand the mechanism of an invention, it is believed that this invention represents a material improvement in medication response prediction. For example, the development of the present invention has resulted in:
1. An increase in predictive accuracy from 0.86 in 2013 to 0.91 as of the date of this filing.
2. Completion of a fourth randomized clinical trial (Interim results published Aug. 28, 2016) demonstrating clinical success of selected QEEG predictors over standard DSM-directed “trial and error” prescription therapy approaches. Iosifescu et al., “The use of the Psychiatric Electroencephalography Evaluation Registry (PEER) to personalize pharmacotherapy” Neuropsychiatric Disease and Treatment 12:2131-2142 (2016).
3. Reductions in Suicidality, as seen in two published studies. For example, a Walter Reed PEER Interactive study of Iraq/Afganistan veteran populations, suicidal ideation decreased 75% more when physicians followed the present method utilizing therapy prioritizations in a PEER Report. Iosifescu et al., “The use of the Psychiatric Electroencephalography Evaluation Registry (PEER) to personalize pharmacotherapy” Neuropsychiatric Disease and Treatment 12:2131-2142 (2016); and DeBattista et al., “The use of referenced-EEG (rEEG) in assisting medication selection for the treatment of depression” Journal of Psychiatric Research 45(1):64-75 (2010).

### VI. Drug Metabolism Screening

In one embodiment, the present invention contemplates a method comprising screening metabolic rates of a plurality of recommended drugs for a specific patient. In one embodiment, the method comprises administering the recommended drug to the patient and creating a pharmacokinetic metabolic profile. In one embodiment, the method comprises taking a biopsy tissue from said patient and using an in vitro metabolic assay using cells from the biopsy tissue.

For example, a metabolic assay may comprise growing and testing eukaryotic cells (e.g., animal or human cells) in a multi-test format. In particular, the assay can provide a complex metabolic profile of animal cells. In addition, the assay would determine effects of recommended drugs on substrate utilization by mammalian cells. Bochner et al., “Methods and kits for obtaining a metabolic profile of living animal cells” U.S. Pat. No. 9,274,101 (herein incorporated by reference).

Other reports suggesting that hepatic microsomal cytochrome P450 (CYP) forms have a role in the metabolism of drugs and other chemicals use primary hepatocyte cultures from humans and experimental animals in an in vitro system for studying the effects of chemicals on CYP forms. Such methods to evaluate CYP form induction in human and rat hepatocytes are cultured in a 96-well plate format. The use of a 96-well plate format permits studies to be performed with relatively small numbers of hepatocytes and obviates the need to harvest cells and prepare subcellular fractions prior to the assay of enzyme activities. The induction of CYP1A and CYP3A forms in human and rat hepatocytes can be determined by measurement of 7-ethoxyresorufin 0-deethylase and testosterone 6b-hydroxylase activities, respectively, whereas 7-benzyloxy-4-trifluoromethylcoumarin (BFC) O-debenzylase can be employed to assess both CYP1A and CYP2B form induction in rat hepatocytes. Lake et al., “In Vitro Assays for Induction of Drug Metabolism” In: Hepatocyte Transplantation, vol. 481, pp 47-58, Anil Dhawan, Robin D. Hughes (eds.) (2009). In particular, CYP-dependent enzyme assays can be performed with human and rat hepatocytes cultured in a 96-well plate format and an assay for hepatocyte protein content that can be used to normalise the results of the CYP-dependent enzyme activity measurements. The use of 7-ethoxyresorufin 0-deethylase activity as a marker for induction of CYP1A forms in human and rat hepatocytes cultured in a 96-well plate format. Testosterone 6b-hydroxylase is well known as a specific marker for CYP3A forms in both human and rodent liver and this activity may also be used as a marker for CYP3A form induction in cultured hepatocytes. Rat hepatocytes has been demonstrated that 7-benzyloxy-4-trifluoromethylcoumarin (BFC) O-debenzylase activity is a good marker for the induction of both CYP1A and CYP2B forms. In human hepatocytes, this enzyme activity may be a marker for CYP1A and possibly also CYP3A forms. When using intact cells, rather than subcellular fractions, for CYP enzyme activity determinations, attention needs to be paid to the possible phase II metabolism of the CYP substrates employed. With the 7-ethoxyresorufin 0-deethylase assay, the resorufin product can be a substrate for cytosolic quinone reductase and is also conjugated with D-glucuronic acid and sulphate. The need for enzymatic deconjugation also applies to the assay of BFC O-debenzylase activity, whereas no enzymatic deconjugation is required for the testosterone 6b-hydroxylase assay. A sulphorhodamine B (SRB) protein assay for hepatocyte protein content may also be performed in a 96-well plate format.

Drug candidate and toxicity screening processes currently rely on results from early-stage in vitro cell-based assays expected to faithfully represent essential aspects of in vivo pharmacology and toxicology. Several in vitro designs have been optimized for high throughput to benefit screening efficiencies, allowing the entire libraries of potential pharmacologically relevant or possible toxin molecules to be screened for different types of cell signals relevant to tissue damage or to therapeutic goals. Creative approaches to multiplexed cell-based assay designs that select specific cell types, signaling pathways and reporters are routine. However, substantial percentages of new chemical and biological entities (NCEs/NBEs) that fail late-stage human drug testing, or receive regulatory “black box” warnings, or that are removed from the market for safety reasons after regulatory approvals all provide strong evidence that in vitro cell-based assays and subsequent preclinical in vivo studies do not yet provide sufficient pharmacological and toxicity data or reliable predictive capacity for understanding drug candidate performance in vivo. Without a reliable translational assay tool kit for pharmacology and toxicology, the drug development process is costly and inefficient in taking initial in vitro cell-based screens to in vivo testing and subsequent clinical approvals. Commonly employed methods of in vitro testing, including dissociated, organotypic, organ/explant, and 3-D cultures, are reviewed here with specific focus on retaining cell and molecular interactions and physiological parameters that determine cell phenotypes and their corresponding responses to bioactive agents. Distinct advantages and performance challenges for these models pertinent to cell-based assay and their predictive capabilities required for accurate correlations to in vivo mechanisms of drug toxicity are compared. Astashkina et al., “A critical evaluation of in vitro cell culture models for high-throughput drug screening” Pharmacology & Therapeutics 134:82-106 (2012).

### EXPERIMENTAL

### Example I

### Hepatic Drug Metabolic In Vitro Assay Protocols

1. The CYP form activities described herein are suitable for use with primary human hepatocytes cultured in a 96-well plate format, employing a seeding density of around 30,000 viable cells/well. The use of a sandwich culture technique (e.g. use of plates coated with a suitable extracellular matrix such as collagen, fibronectin or Matrigel and the attached hepatocytes then overlaid with extracellular matrix) is recommended. Human hepatocytes are normally cultured in control medium for 1-3 days before being treated with CYP form inducers. To study the induction of CYP forms, primary hepatocyte cultures are treated with the test compounds (i.e. the compounds under investigation) and reference items (see below) for a suitable period (e.g. 2 or 3 days). Normally the culture medium is changed at 24 h intervals and replaced with fresh medium containing the test compounds and reference items. Test compounds and reference items may be added to the culture medium in DMSO

2. When employing 96-well plates, replicates are normally performed for both cells cultured in control medium and for cells treated with the test compounds and reference items. For 7-ethoxyresorufin 0-deethylase and BFC O-debenzylase fluorescent assays, up to 12 wells/plate should be controls (i.e. hepatocytes cultured in control medium containing the DMSO solvent) and up to 6 wells/plate for each concentration of each test compound and reference item. With the radiometric testosterone 6b-hydroxylase assay, it may be necessary to pool two or three wells for each control and treatment in order to provide a sufficient volume of incubation medium for HPLC analysis.

3. For all assays, suitable blanks should be run in parallel with the treatment of the hepatocyte preparations. These consist of incubations in 96-well plates containing the overlay (e.g. collagen or Matrigell) and control medium but no hepatocytes. For the two fluorescent assays, eight blank wells are normally sufficient, whereas for the radiometric assay up to four wells or four pools of two or more wells may be required.

4. To assess the functional viability of human and rat hepatocyte preparations for CYP form induction studies, the use of reference items is recommended. Suitable reference item concentrations (see Note 3) are as follows:
(a) For CYP1A form induction in human hepatocytes use 2 and 10 mM BNF and for rat hepatocytes use 0.2 and 2 mM BNF.
(b) For CYP2B form induction in rat hepatocytes use 200 and 500 mM NaPB.
(c) For CYP3A form induction in human hepatocytes use 2 and 10 mMRIF. Studies may also be conducted with 200 and 500 mM NaPB.
(d) For CYP3A form induction in rat hepatocytes use 2 and 20 mM PCN.

### Example II

### Assay of 7-Ethoxyresorufin O-Deethylase Activity

1. Prepare sufficient 7-ethoxyresorufin substrate solution (added at 100 ml/well) for all wells and plates to be assayed, by thawing aliquots stored at −208 C of 2 mM 7-ethoxyresorufin in DMSO and 20 mM dicumarol in DMSO. Add 4 ml/ml 2 mM 7-ethoxyresorufin and 0.5 ml/ml 20 mM dicumarol in DMSO per millilitre of RPMI 1640 (phenol red free) medium at 37° C. Mix the substrate solution (final concentrations 7-ethoxyresorufin 8 mM and dicumarol 10 mM) with a vortex mixer and return to the incubator.

2. At the end of the treatment period with the test compounds and the reference items, the medium is removed and the cells washed with 200 ml/well of RPMI 1640 (phenol red free) medium at 37° C. Return the plates to the incubator.

3. Remove the RPMI 1640 (phenol red free) wash medium from each plate and quickly add 100 ml/well of the 8 mM 7-ethoxyresorufin/10 mM dicumarol substrate solution to each well and mix the plates for 5 s on a gyratory shaker.

4. Return the plates to the tissue culture incubator and incubate for a suitable period (e.g., approximately 30 min) at 37° C.

5. At the end of the incubation period, mix the plates on a gyratory shaker for 5 s and remove a 75 ml aliquot of the medium from each well into a “V”-bottomed 96-well plate and store at −808 C prior to analysis.

6. Thaw the “V”-bottomed 96-well plates and add 10 ml/well 0.5M sodium acetate buffer pH5.0 and 15 ml/well of the β-glucuronidase/sulphatase solution (see Section 2.2) to all wells, mix the plates for 5 s on a gyratory shaker and incubate for 2 h at 378 C.

7. Prepare a 2 mM resorufin standard by thawing an aliquot of 2 mM resorufin in DMSO and diluting 10 ml to a final volume of 10 ml with RPMI 1640 (phenol red free) medium. Set up a standard curve by adding 0 (blank), 5, 10, 15, 20, 25, 30, 40 and 50 ml aliquots of the 2 mM resorufin standard to a “V”-bottomed 96-well plate (for the standard curve use eight replicate wells for each resorufin concentration) and add 25-75 ml/well of RPMI 1640 (phenol red free) medium so that each well has a final volume of 75 ml. Add 10 ml/well 0.5M sodium acetate buffer pH 5.0 and 15 ml/well of the b-glucuronidase/sulphatase solution to all wells, mix the plates for 5 s on a gyratory shaker and incubate for 2 h at 37° C.

8. At the end of the incubation period, add 100 ml of 0.25 M Tris in 60% (v/v) ACN to all wells and mix the plates on a gyratory shaker for 15 s. Transfer 150 ml from each well into a flat-bottomed white polystyrene 96-well plate. Set up a fluorescence spectrophotometer with a 96-well plate reader and determine the fluorescence of each well at excitation and 52 Lake et al. emission wavelengths of 535 and 582 nm, respectively.

9. For the resorufin standard curve, subtract the mean fluorescence of the blank wells (no resorufin standard) and plot fluorescence units against picomole of resorufin added (in the 150 ml sample analysed, the resorufin standards range from 7.5 to 75 pmol).

10. For the hepatocyte samples, subtract the mean fluorescence of the blank wells (i.e. the wells containing no hepatocytes) from the test wells and using the standard curve (see above) determine the picomole resorufin formed per well. By allowing for the incubation time, the results are expressed either as picomole resorufin formed per minute per number of cells per well or with the hepatocyte protein content of each well as picomole resorufin formed per minute per microgram hepatocyte protein.

### Example III

### Assay of BFC O-Debenzylase Activity

1. Prepare sufficient BFC substrate solution (added at 100 ml/well) for all wells and plates to be assayed, by thawing aliquots stored at −20° C. of 12.5 mM BFC. Add 4 ml/ml 12.5 mM BFC per millilitre of RPMI 1640 (phenol red free) medium at 37° C. Mix the substrate solution (final BFC concentration 50 mM) with a vortex mixer and return to the incubator.

2. At the end of the treatment period with the test compounds and the reference items, the medium is removed and the cells washed with 200 ml/well of RPMI 1640 (phenol red free) medium at 37° C. Return the plates to the incubator.

3. Remove the RPMI 1640 (phenol red free) wash medium from each plate and quickly add 100 ml/well of the 50 mM BFC substrate solution to each well and mix the plates for 5 s on a gyratory shaker.

4. Return the plates to the tissue culture incubator and incubate for a suitable period (e.g. 20 min for rat hepatocytes) at 37° C.

5. At the end of the incubation period, mix the plates on a gyratory shaker for 5 s and remove a 75 ml aliquot of the medium from each well into a “V”-bottomed 96-well plate and store at −80° C. prior to analysis.

6. Thaw the “V”-bottomed 96-well plates and add 10 ml/well 0.5 M sodium acetate buffer pH 5.0 and 15 ml/well of the b-glucuronidase/sulphatase solution to all wells, mix the plates for 5 s on a gyratory shaker and incubate for 2 h at 37° C.

7. Prepare a 6.667 mM HFC standard by thawing an aliquot of 0.6667 mM HFC in DMSO and diluting 100 ml to a final volume of 10 ml with RPMI 1640 (phenol red free) medium. Set up a standard curve by adding 0 (blank), 5, 10, 15, 20, 25, 30, 40 and 50 ml aliquots of the 6.667 mMHFC standard to a “V”-bottomed 96-well plate (for the standard curve use 8 replicate wells for each HFC concentration) and add 25-75 ml/well of RPMI 1640 (phenol red free) medium so that each well has a final volume of 75 ml. Add 10 ml/well 0.5M sodium acetate buffer pH 5.0 and 15 ml/well of the β-glucuronidase/sulphatase solution to all wells, mix the plates for 5 s on a gyratory shaker and incubate for 2 h at 37° C.

8. At the end of the incubation period, add 100 ml of 0.25 M Tris in 60% (v/v) ACN to all wells and mix the plates on a gyratory shaker for 15 s. Transfer 150 ml from each well into a flat-bottomed white polystyrene 96-well plate. Set up a fluorescence spectrophotometer with a 96-well plate reader and determine the fluorescence of each well at excitation and emission wavelengths of 410 and 510 nm, respectively.

9. For the HFC standard curve, subtract the mean fluorescence of the blank wells (no HFC standard) and plot fluorescence units against picomole of HFC added (in the 150 ml sample analysed the HFC standards range from 25 to 250 pmol).

10. For the hepatocyte samples, subtract the mean fluorescence of the blank wells (i.e. the wells containing no hepatocytes) from the test wells and using the standard curve (see above) determine the picomole HFC formed per well. By allowing for the incubation time, the results are expressed either as picomole HFC formed per minute per number of cells per well or with the hepatocyte protein content of each well as picomole HFC formed per minute per milligram hepatocyte protein.

### Example IV

### Assay of Testosterone 6b-Hydroxylase Activity

1. Prepare sufficient 250 mM[4-14 C]testosterone substrate solution to add at 100 ml/well with each well receiving 0.4 mCi radioactivity. For example, 10 ml of substrate solution will contain 2.5 mmol testosterone and 40 mCi radioactivity. Add 40 mCi of stock [4-14 C] testosterone to a tapered glass tube and remove the solvent with a stream of nitrogen. Then add 10 ml of DMSO containing unlabelled testosterone so that the tube contains a total of 2.5 mmol of labelled and unlabelled testosterone. For a specific activity of 54 mCi/mmol, the unlabelled testosterone substrate solution will be 175.93 mM. Vortex the tube contents and transfer the DMSO solvent to 10 ml of RPMI 1640 (phenol red free) medium at 378 C and mix well with a vortex mixer. Add 10 ml of DMSO to the tapered glass tube, vortex the tube contents and transfer to the RPMI 1640 (phenol red free) medium at 37° C. Repeat with two further 10 ml and one 5 ml washes of DMSO. Mix the 250 mM 54 Lake et al. [4-14 C]testosterone substrate with a vortex mixer and return to the incubator.

2. At the end of the treatment period with the test compounds and the reference items, the medium is removed and the cells washed with 200 ml/well of RPMI 1640 (phenol red free) medium at 37° C. Return the plates to the incubator.

3. Remove the RPMI 1640 (phenol red free) wash medium from each plate and quickly add 100 ml/well of the 250 mM [4-14 C]testosterone substrate solution to each well and mix the plates for 5 s on a gyratory shaker.

4. Return the plates to the tissue culture incubator and incubate at 37° C. for a suitable period (e.g. 30 and 20 min for human and rat hepatocytes, respectively) at 37° C.

5. At the end of the incubation period, mix the plates on a gyratory shaker for 5 s and remove the medium from all wells into Eppendorf tubes, pooling wells as required (see Section 3.1). Store the tubes at −80° C. prior to analysis.

6. Thaw the samples and analyse aliquots by HPLC, employing Supelcosil-5 LC-18 (Sigma-Aldrich) and mobile phases of ACN (A), ultrapure water (B), methanol (C) and 10% (v/v) acetic acid in ultrapure water (D). Elution is achieved at a flow rate of 2 ml/min starting with 12% A, 73% B, 10% C and 5% D for 10 min, changing to 12% A, 67% B, 16% C and 5% D over 14.2 min, changing to 14% A, 81% C and 5% D over 1 min, holding at 14% A, 81% C and 5% D for 4 min, changing to 12% A, 73% B, 10% C and 5% D over 0.8 min, holding at 12% A, 73% B, 10% C and 5% D for 4 min and equilibrating at 12% A, 73% B, 10% C and 5% D for 4 min before the next injection. Retention times of testosterone and 6b-hydroxytestosterone are approximately 18 and 14 min, respectively. Formation of 6b-hydroxytestosterone is quantified by radiometric detection.

7. The amount of 6b-hydroxytestosterone formed in the sample less any material present in the blank (no hepatocytes) incubations is determined as a percentage of the substrate added (25 nmol per well). By allowing for the incubation time, the results are expressed either as picomole 6b-hydroxytestosterone formed per minute per number of cells per well or with the hepatocyte protein content of each well as picomole 6b hydroxytestosterone formed per minute per milligram hepatocyte protein.

We claim:

1. A method, comprising:
a) collecting an electroencephalogram and a plurality of cells from a patient exhibiting at least one symptom of a diagnosed psychiatric disorder;
b) converting said electroencephalogram into at least one quantitative electroencephalographic (QEEG) feature variable;
c) identifying at least one genotype in said plurality of cells;
d) comparing said at least one QEEG feature variable to a first database to create a first therapy list prioritized according to a first predicted efficacy score, said first therapy list comprising a first recommended therapy;
e) comparing said at least one genotype to a second database to create a second therapy list prioritized according to a second predicted efficacy score, said second therapy list comprising a second recommended therapy;
f) matching said first therapy list and said second therapy list to create a final therapy list prioritized according to a combined first and second efficacy score, said final therapy list comprising a final recommended therapy; and
g) administering said final recommended therapy to said patient under conditions such that said at least one symptom is reduced, wherein said selected therapy comprises a combined first and second efficacy score that is within a preferred range.

2. The method of claim 1, wherein said final recommended therapy is different from said first recommended therapy and said second recommended therapy.

3. The method of claim 1, wherein said first recommended therapy and said second recommended therapy are the same.

4. The method of claim 1, wherein said first recommended therapy and said second recommended therapy are different.

5. A method, comprising:
a) collecting an electroencephalogram and a plurality of cells from a patient exhibiting at least one symptom of a diagnosed psychiatric disorder;
b) converting said electroencephalogram into at least one quantitative electroencephalographic (QEEG) feature variable;
c) comparing said at least one QEEG feature variable to a first database to identify a prioritized list of recommended drugs;
d) processing said prioritized list of recommended drugs with an in vitro enzyme metabolism assay using said plurality of cells to identify a list of said recommended drugs prioritized by metabolic rate;
e) selecting a preferred recommended drug by identification of a non-metabolic drug biomarker in said plurality of cells that matches at least one drug on said metabolic rate prioritized list of recommended drugs;
f) administering said preferred recommended drug to said patient under conditions such that said at least one symptom is reduced.

6. The method of claim 5, wherein said non-metabolic drug biomarker is a blood based biomarker.

7. The method of claim 5, wherein said non-metabolic drug biomarker is a cell based biomarker.

8. A method, comprising:
a) collecting an electroencephalogram and a plurality of cells from a patient exhibiting at least one symptom of a diagnosed psychiatric disorder;
b) converting said electroencephalogram into at least one quantitative electroencephalographic (QEEG) feature variable, said QEEG feature variable having a predetermined drug efficacy predictive value;
c) identifying at least one genotype in said plurality of cells, said at least one genotype having a predetermined drug efficacy predictive value;
d) combining said QEEG feature variable predetermined drug efficacy predictive value and said at least one genotype predetermined drug efficacy predictive value to create a list of recommended drugs prioritized by an efficacy score; and
e) administering at least one of said recommended drugs to said patient under conditions such that said at least one symptom is reduced, wherein said efficacy score of said selected drug is within a preferred range.

9. The method of claim 8, wherein said at least one genotype encodes a non-metabolic drug efficacy predictor.

10. The method of claim 8, wherein said at least one genotype encodes a metabolic drug efficacy predictor.

11. The method of claim 10, wherein said method further comprises measuring a metabolic rate of said at least one said recommended drugs using said metabolic drug efficacy predictor.

12. A method, comprising:
a) collecting an electroencephalogram and a plurality of cells from a patient exhibiting at least one symptom of a diagnosed psychiatric disorder;
b) converting said electroencephalogram into at least one quantitative electroencephalographic (QEEG) feature variable;
c) comparing said at least one QEEG feature variable to a first database to identify a prioritized list of recommended drugs;
d) processing said prioritized list of recommended drugs with at least one metabolic genotype using said plurality of cells to identify a list of said recommended drugs prioritized by metabolic rate;
e) selecting a preferred recommended drug by identification of a non-metabolic drug biomarker in said plurality of cells that matches at least one drug on said metabolic rate genotype prioritized list of recommended drugs;
f) administering said preferred recommended drug to said patient under conditions such that said at least one symptom is reduced.

13. The method of claim 12, wherein said plurality of cells is derived from a patient biopsy.
